(19) United States
(12) Patent Application Publication
(10) Pub. No.: US 2026/0151511 A1
(43) Pub. Date: Jun. 4, 2026

(54) TREATMENT OF DISEASES ASSOCIATED WITH PATHOLOGIC NEURONAL CELLS

(71) Applicant: Modulight Bio Ltd., Ramat-Gan (IL)

(72) Inventors: Yotam ELDAR, Boston, MA (US); Ofir LEVI, Tel Aviv (IL)

(73) Assignee: Modulight Bio Ltd., Ramat-Gan (IL)

(21) Appl. No.: 19/460,438

(22) Filed: Jan. 27, 2026

Related U.S. Application Data
(63) Continuation of application No. PCT/IL2024/050742, filed on Jul. 26, 2024.
(60) Provisional application No. 63/529,183, filed on Jul. 27, 2023.

(51) Int. Cl. A61K 48/00; A61K 38/17; A61K 41/00; A61P 25/04; A61P 25/06; A61P 25/08; A61P 25/16; A61P 25/20

(52) U.S. Cl. CPC A61K 48/0058; A61K 38/1787; A61K 41/00; A61P 25/04; A61P 25/06; A61P 25/08; A61P 25/16; A61P 25/20; C12N 2750/14143

(57) ABSTRACT

Methods of treating diseases associated with pathologic neuronal cells are provided. Accordingly, there is provided a method of treating a disease associated with pathologic neuronal cells characterized by aberrant excitability comprising: (a) administering into a first neural region of the subject a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; and (b) exposing a second neural region of said subject to light in a wavelength that activates said bistable type II opsin, wherein said second neural region comprises axon or dendrite terminals of said pathologic neuronal cells associated with a symptom of said disease, and wherein said first neural region and said second neural region are distinct.

### RELATED APPLICATIONS

This application is a Continuation of PCT Patent Application No. PCT/IL2024/050742 having International filing date of Jul. 26, 2024 which claims the benefit of priority under 35 USC § 119(e) of U.S. Provisional Patent Application No. 63/529,183 filed on Jul. 27, 2023. The contents of the above applications are all incorporated by reference as if fully set forth herein in their entirety.

### SEQUENCE LISTING STATEMENT

The XML file, entitled 105020SequenceListing.xml, created on Jan. 27, 2026, comprising 57,172 bytes, submitted concurrently with the filing of this application is incorporated herein by reference.

### FIELD AND BACKGROUND OF THE INVENTION

The present invention, in some embodiments thereof, relates to treatment of diseases associated with pathologic neuronal cells.

The human nervous system relies on the proper functioning of neuronal cells for essential cognitive, motor, sensory, and emotional processes. However, when neuronal cells undergo pathological changes, it can lead to severe impairments and the manifestation of various diseases, including, for example, Parkinson's disease, dystonia, essential tremor, epilepsy and neuropathic pain.

Current treatments for these diseases, such as specific drugs and surgical and ablation interventions have limitations in terms of efficacy, adverse effects, and invasiveness. Deep-brain stimulation (DBS), for example, has been shown to be effective for epilepsy, Parkinson's disease and several other movement disorders [e.g. Salanova V. et al.; SANTÉ Study Group. (2021) Epilepsia. 62(6): 1306-1317; Benabid A L et al. Lancet. 1991 Feb. 16; 337(8738):403-6; and Müller U J et al. Ann N Y Acad Sci. 2013 April; 1282: 119-28]. However, the specificity of electrical DBS is limited by the non-specific effects of the stimulation currents on diverse neurons and glial cells located at the site of stimulation or adjacent to it causing many adverse effects.

In recent years, the development of cellular perturbation tools based on light sensitive proteins has resulted in a technology called optogenetics, referring to the integration of genetic and optical control to achieve gain- or loss-of-function of precisely defined events within specified cell types of living tissues. This technique typically involves the use of proteins called opsins, a major class of light-sensitive proteins that can be found across all kingdoms of life and serve a diverse range of functions. Opsins can be divided into two groups, while both types are seven-transmembrane-domain proteins belonging to the G protein-coupled receptor (GPCR) superfamily, type I opsins (e.g. the microbial opsins) are ion channels or proton/ion pumps and thus are activated by light directly, while type II opsins activate G-proteins, which then activate effector enzymes that produce metabolites to e.g. open ion channels. Both types of opsins were suggested for optogenetic approaches (see e.g. International Patent Application Publication nos. WO2013090356 and WO2020/188572; Rost et al. Nat Neurosci (2022) 25(8): 984-998; and Paz et al. Nat Neurosci (2013)16(1): 64-70).

### SUMMARY OF THE INVENTION

According to an aspect of some embodiments of the present invention there is provided a method of treating a disease associated with pathologic neuronal cells characterized by aberrant excitability in a subject in need thereof, the method comprising:
(a) administering into a first neural region of the subject a therapeutically effective amount of a polynucleotide encoding a bistable type II opsin attached to a heterologous ER export signal and/or membrane trafficking signal which enables trafficking of the bistable type II opsin to axon and/or dendrite terminals, wherein the first neural region comprises cell bodies of the pathologic neuronal cells; and
(b) exposing a second neural region of the subject to light in a wavelength that activates the bistable type II opsin, wherein the second neural region comprises axon or dendrite terminals of the pathologic neuronal cells associated with a symptom of the disease,
wherein the first neural region and the second neural region are distinct,
thereby treating the disease in the subject.

According to some embodiments of the invention, the aberrant excitability comprises hyper-excitability

According to some embodiments of the invention, the aberrant excitability comprises mis-synchronization.

According to some embodiments of the invention, the aberrant excitability comprises spatial or temporal aberrancy.

According to some embodiments of the invention, the disease is selected from the group consisting of epilepsy, Parkinson's disease, essential tremor, pain, spasticity, Achalasia, obsessive-compulsive disorder (OCD), addiction, appetite disorder, autoimmune disorder, narcolepsy and insomnia.

According to some embodiments of the invention, when the disease is epilepsy the first neural region is not a thalamus nucleus.

According to some embodiments of the invention, the disease, the first neural region and the second neural region are as listed in Table 1.

According to some embodiments of the invention, the administering is by a stereotactic injection.

According to some embodiments of the invention, the polynucleotide is packed in a viral vector.

According to some embodiments of the invention, a nucleic acid sequence encoding the bistable type II opsin is codon optimized to mammalian expression.

According to some embodiments of the invention, the bistable type II opsin is selected from the group consisting of OPN3, OPN4, OPN5, LcPP, DrPP2, TrPP2, parapinopsin, PdCO, TMT and peropsin.

According to some embodiments of the invention, the bistable type II opsin is OPN3.

According to some embodiments of the invention, the OPN3 is mosquito OPN3 (MosOpn3).

According to some embodiments of the invention, the ER export signal and/or the membrane trafficking signal is of a protein expressed in neuronal cells.

According to some embodiments of the invention, the ER export signal and/or the membrane trafficking signal is of a Kir2.1 polypeptide.

According to some embodiments of the invention, an amino acid sequence of the ER export signal comprises SEQ ID NO: 2.

According to some embodiments of the invention, an amino acid sequence of the membrane trafficking signal comprises SEQ ID NO: 1.

According to some embodiments of the invention, the exposing is effected at least 6 weeks following the administering.

According to some embodiments of the invention, the exposing is effected at least 8 weeks following the administering.

According to some embodiments of the invention, the exposing comprises repeated illumination independent of detection of an acute symptom of the disease.

According to some embodiments of the invention, the exposing is effected upon detection of an acute symptom of the disease.

According to some embodiments of the invention, the detection is by electrophysiological recording.

According to some embodiments of the invention, the exposing is effected using a skull-mounted or intracranial device.

According to some embodiments of the invention, the exposing is effected using a device implanted along the spine.

According to some embodiments of the invention, the exposing is effected using a device external to the subject.

According to some embodiments of the invention, the device allows electrophysiological recording and illumination.

According to some embodiments of the invention, the wavelength is 450-650 nm.

According to some embodiments of the invention, the wavelength is 350-670 nm.

1. A method of treating a disease associated with pathologic neuronal cells characterized by aberrant excitability in a subject in need thereof, the method comprising:
(a) administering into a first neural region of the subject a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; and
(b) exposing a second neural region of said subject to light in a wavelength that activates said bistable type II opsin, wherein said second neural region comprises axon or dendrite terminals of said pathologic neuronal cells associated with a symptom of said disease,
wherein said first neural region and said second neural region are distinct, and
wherein when said disease is epilepsy said first neural region is not a thalamus nucleus,
thereby treating the disease in the subject.

2. A method of treating a disease associated with pathologic neuronal cells characterized by aberrant excitability in a subject in need thereof having been administered into a first neural region a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; the method comprising exposing a second neural region of said subject to light in a wavelength that activates said bistable type II opsin, wherein said second neural region comprises axon or dendrite terminals of said pathologic neuronal cells associated with a symptom of said disease, wherein said first neural region and said second neural region are distinct, and wherein when said disease is epilepsy said first neural region is not a thalamus nucleus, thereby treating the disease in the subject.

3. A composition comprising a polynucleotide encoding a bistable type II opsin attached to a heterologous ER export signal and/or membrane trafficking signal which enables trafficking of said bistable type II opsin to axon and/or dendrite terminals, for use in treating a disease associated with pathologic neuronal cells characterized by aberrant excitability in a subject in need thereof, the composition being used in combination with a device for exposing a neural region of said subject to light, wherein the treatment is characterized by:
(a) administration of said composition into a first neural region of the subject, wherein said first neural region comprises cell bodies of said pathologic neuronal cells; and
(b) exposure of a second neural region of said subject to light in a wavelength that activates said bistable type II opsin, wherein said second neural region comprises axon or dendrite terminals of said pathologic neuronal cells associated with a symptom of said disease,
wherein said first neural region and said second neural region are distinct, and
wherein when said disease is epilepsy said first neural region is not a thalamus nucleus.

4. A composition comprising a polynucleotide encoding a bistable type II opsin attached to a heterologous ER export signal and/or membrane trafficking signal which enables trafficking of said bistable type II opsin to axon and/or dendrite terminals, for use in treating a disease associated with pathologic neuronal cells characterized by aberrant excitability in a subject in need thereof, wherein the treatment is characterized by:
(a) administration of said composition into a first neural region of the subject, wherein said first neural region comprises cell bodies of said pathologic neuronal cells; and
(b) activation of said bistable type II opsin in a second neural region of said subject by light, wherein said second neural region comprises axon or dendrite terminals of said pathologic neuronal cells associated with a symptom of said disease,
wherein said first neural region and said second neural region are distinct, and
wherein when said disease is epilepsy said first neural region is not a thalamus nucleus.

5. A composition comprising a polynucleotide encoding a bistable type II opsin attached to a heterologous ER export signal and/or membrane trafficking signal which enables trafficking of said bistable type II opsin to axon and/or dendrite terminals, for use in treating a disease associated with pathologic neuronal cells characterized by aberrant excitability in a subject in need thereof, wherein the composition is formulated for administration to a neural region comprising cell bodies of said pathologic neuronal cells and not to terminals of said cell bodies, wherein when said disease is epilepsy said neural region is not a thalamus nucleus.

6. The method of any one of claims 1-2 or the composition for use of any one of claims 3-5, wherein said aberrant excitability comprises hyper-excitability.

7. The method of any one of claims 1-2 or the composition for use of any one of claims 3-5, wherein said aberrant excitability comprises mis-synchronization.

8. The method of any one of claims 1-2 or the composition for use of any one of claims 3-5, wherein said aberrant excitability comprises spatial or temporal aberrancy.

9. The method or the composition for use of any one of claims 1-8, wherein said disease is selected from the group consisting of epilepsy, Parkinson's disease, essential tremor, pain, spasticity, Achalasia, obsessive-compulsive disorder (OCD), addiction, appetite disorder, autoimmune disorder, narcolepsy and insomnia.

10. The method or the composition for use of any one of claims 1-8, wherein said disease, said first neural region and said second neural region are as listed in Table 1.

11. The method or the composition for use of any one of claims 1-10, wherein said administering or said administration is by a stereotactic injection.

12. The method or the composition for use of any one of claims 1-11, wherein said polynucleotide is packed in a viral vector.

13. The method or the composition for use of any one of claims 1-12, wherein a nucleic acid sequence encoding said bistable type II opsin is codon optimized to mammalian expression.

14. The method or the composition for use of any one of claims 1-13, wherein said bistable type II opsin is selected from the group consisting of OPN3, OPN4, OPN5, LcPP, DrPP2, TrPP2, parapinopsin, PdCO, TMT and peropsin.

15. The method or the composition for use of any one of claims 1-13, wherein said bistable type II opsin is OPN3.

16. The method or the composition for use of claim 15, wherein said OPN3 is mosquito OPN3 (MosOpn3).

17. The method or the composition for use of any one of claims 1-16, wherein said ER export signal and/or said membrane trafficking signal is of a protein expressed in neuronal cells.

18. The method or the composition for use of any one of claims 1-17, wherein said ER export signal and/or said membrane trafficking signal is of a Kir2.1 polypeptide.

19. The method or the composition for use of any one of claims 1-18, wherein an amino acid sequence of said ER export signal comprises SEQ ID NO: 2.

20. The method or the composition for use of any one of claims 1-19, wherein an amino acid sequence of said membrane trafficking signal comprises SEQ ID NO: 1.

21. The method of any one of claims 1-2 and 6-20, wherein said exposing is effected at least 6 weeks following said administering.

22. The method of any one of claims 1-2 and 6-20, wherein said exposing is effected at least 8 weeks following said administering.

23. The method of any one of claims 1-2 and 6-22, wherein said exposing comprises repeated illumination independent of detection of an acute symptom of said disease.

24. The method of any one of claims 1-2 and 6-22, wherein said exposing is effected upon detection of an acute symptom of said disease.

25. The method of claim 24, wherein said detection is by electrophysiological recording.

26. The method of any one of claims 1-2 and 6-25, wherein said exposing is effected using a skull-mounted or intracranial device.

27. The method of any one of claims 1-2 and 6-25, wherein said exposing is effected using a device implanted along the spine.

28. The method of any one of claims 1-2 and 6-25, wherein said exposing is effected using a device external to the subject.

29. The composition for use of any one of claims 3-4 and 6-20, wherein said exposure or said activation is effected at least 6 weeks following said administration.

30. The composition for use of any one of claims 3-4 and 6-20, wherein said exposure or said activation is effected at least 8 weeks following said administration.

31. The composition for use of any one of claims 3-4, 6-20 and 29-30, wherein said exposure or said activation comprises repeated illumination independent of detection of an acute symptom of said disease.

32. The composition for use of any one of claims 3-4, 6-20 and 29-30, wherein said exposure or said activation is effected upon detection of an acute symptom of said disease.

33. The composition for use of claim 32, wherein said detection is by electrophysiological recording.

34. The composition for use of any one of claims 3-4, 6-20 and 29-33, wherein said exposure or said activation is effected using a skull-mounted or intracranial device.

35. The composition for use of any one of claims 3-4, 6-20 and 29-33, wherein said exposure or said activation is effected using a device implanted along the spine.

36. The composition for use of any one of claims 3-4, 6-20 and 29-33, wherein said exposure or said activation is effected using a device external to the subject.

37. The method of any one of claims 26-28 or the composition for use of any one of claims 34-36, wherein said device allows electrophysiological recording and illumination.

38. The method or the composition for use of any one of claims 1-37, wherein said wavelength is 450-650 nm.

39. The method or the composition for use of any one of claims 1-37, wherein said wavelength is 350-670 nm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an experimental timeline for treatment of epilepsy.

FIG. 2 is a schematic representation of an experimental timeline for treatment of Parkinson's disease.

FIG. 3 is a schematic representation of an experimental timeline for treatment of trigeminal pain.

FIG. 4 is a schematic representation of an experimental timeline for treatment of peripheral pain.

FIG. 5 is a schematic representation of an experimental timeline for treatment of sleep disorders.

FIG. 6 is a schematic representation of an experimental Timeline for treatment of essential tremor.

FIG. 7 is a schematic representation of major circuitry nodes in Essential Tremor. Also shown are injection and illumination targets in the eOPN3 treatment.

FIG. 8 shows a histology slide depicting a mouse brain region including the cerebellar cortex and Deep Cerebellar Nucleus areas. Red colored nano-beads were injected into the cerebellar cortex, demonstrating the eOPN3 injection target. Green colored nano-beads were injected into the Deep Cerebellar Nucleus, demonstrating the optical fibers implant and illumination.

FIGS. 9A-B show spectrograms derived from accelerometer recordings, demonstrating the effect of illumination of Deep Cerebellar Nucleus areas following injection of eOPN3 to the cerebellar cortex in the mouse essential tremor model. Black lines depict base line recording start, green lines depict illumination start, and grey lines depict recovery start. FIG. 9B is a zoom-in on 1^{st} illumination cycle, showing tremor start after Harmaline injection, stops after illumination of the DCN, and returns 1-2 minutes after light is turned off.

### DESCRIPTION OF SPECIFIC EMBODIMENTS OF THE INVENTION

The present invention, in some embodiments thereof, relates to treatment of diseases associated with pathologic neuronal cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst reducing specific embodiments of the present invention to practice, the present inventors have now uncovered that bistable type II opsins can be used as a treatment modality for disease associated with pathologic neuronal cells, while utilizing the neuronal projections associated with symptoms of the disease by administering the bistable type II opsins in one place and illuminating in a wavelength that activates said bistable type II opsin in a distinct place than the administration place. Specific embodiments suggest that this method may allow better efficacy due to the specificity of expression in chosen cell type [e.g. excitatory (e.g. glutamatergic) neurons], while reducing adverse effects due to the precision in modulation and/or reducing invasiveness by eliminating the need for indwelling electrodes [thereby reducing surgical side effects (e.g. infections, bleeding, electrode migration, etc.)].

Thus, according to an aspect of the present invention, there is provided a method of treating a disease associated with pathologic neuronal cells characterized by aberrant excitability in a subject in need thereof, the method comprising:
(a) administering into a first neural region of the subject a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; and
(b) exposing a second neural region of said subject to light in a wavelength that activates said bistable type II opsin, wherein said second neural region comprises axon or dendrite terminals of said pathologic neuronal cells associated with a symptom of said disease,
wherein said first neural region and said second neural region are distinct,
thereby treating the disease in the subject.

According to an additional or an alternative aspect of the present invention, there is provided a method of treating a disease associated with pathologic neuronal cells characterized by aberrant excitability in a subject in need thereof having been administered into a first neural region a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; the method comprising exposing a second neural region of said subject to light in a wavelength that activates said bistable type II opsin, wherein said second neural region comprises axon or dendrite terminals of said pathologic neuronal cells associated with a symptom of said disease, wherein said first neural region and said second neural region are distinct, thereby treating the disease in the subject.

According to an additional or an alternative aspect of the present invention, there is provided a composition comprising a polynucleotide encoding a bistable type II opsin attached to a heterologous ER export signal and/or membrane trafficking signal which enables trafficking of said bistable type II opsin to axon and/or dendrite terminals, for use in treating a disease associated with pathologic neuronal cells characterized by aberrant excitability in a subject in need thereof, the composition being used in combination with a device for exposing a neural region of said subject to light, wherein the treatment is characterized by:
(a) administration of said composition into a first neural region of the subject, wherein said first neural region comprises cell bodies of said pathologic neuronal cells; and
(b) exposure of a second neural region of said subject to light in a wavelength that activates said bistable type II opsin, wherein said second neural region comprises axon or dendrite terminals of said pathologic neuronal cells associated with a symptom of said disease,
wherein said first neural region and said second neural region are distinct.

According to an additional or an alternative aspect of the present invention, there is provided a composition comprising a polynucleotide encoding a bistable type II opsin attached to a heterologous ER export signal and/or membrane trafficking signal which enables trafficking of said bistable type II opsin to axon and/or dendrite terminals, for use in treating a disease associated with pathologic neuronal cells characterized by aberrant excitability in a subject in need thereof, wherein the treatment is characterized by:
(a) administration of said composition into a first neural region of the subject, wherein said first neural region comprises cell bodies of said pathologic neuronal cells; and
(b) activation of said bistable type II opsin in a second neural region of said subject by light, wherein said second neural region comprises axon or dendrite terminals of said pathologic neuronal cells associated with a symptom of said disease,
wherein said first neural region and said second neural region are distinct.

According to an additional or an alternative aspect of the present invention, there is provided a composition comprising a polynucleotide encoding a bistable type II opsin attached to a heterologous ER export signal and/or membrane trafficking signal which enables trafficking of said bistable type II opsin to axon and/or dendrite terminals, for use in treating a disease associated with pathologic neuronal cells characterized by aberrant excitability in a subject in need thereof, wherein the composition is formulated for administration to a neural region comprising cell bodies and not to terminals of said cell bodies of said pathologic neuronal cells.

As used herein, the term “treating” or “treatment” refers to inhibiting, preventing or arresting the development of a pathology (disease, disorder or medical condition i.e. a disease associated with pathologic neuronal cells characterized by aberrant excitability) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology, as further described infra.

As used herein, the term “subject” includes mammals, e.g., human beings at any age and of any gender who suffers from the pathology (disease, disorder or medical condition i.e. a disease associated with pathologic neuronal cells characterized by aberrant excitability).

According to specific embodiments, the subject is a human subject.

According to specific embodiments, the subject is resistant to treatment with drugs or for treating the disease or suffers from adverse effects associated with drugs or other modalities known for the treatment of the disease [e.g. Deep-brain stimulation (DBS)] that prevent their use.

However, according specific embodiments, the methods disclosed herein can be used in combination with other established or experimental therapeutic regimen to treat the disease including, but not limited to, Levetiracetam, Valproic acid (or other Anti-Seizure Medications), Levodopa, Amantadine (Or other Parkinson's medications), Gabapentin, Opiates (or other pain medications), Sodium Oxybate (Gamma Hydroxybutyrate), Modafinil (Or other sleep disorders medication).

As used herein, the term “aberrant excitability” includes hyper-excitability, mis-synchronization and/or spatial or temporal aberrancy. The term “aberrant excitability” does not refer to hypo-excitability or lack of excitation. All these are with respect to the excitability of a normal healthy neuronal tissue of the same spatial location, gender and age.

As used herein, the term “disease associated with pathologic neuronal cells characterized by aberrant excitability” refers to a disease wherein pathological neuronal cells characterized by aberrant excitability drive onset or progression of the disease or symptoms thereof.

It should be emphasized that the term “pathologic” in the context of neuronal cells refers to the neuronal activity (i.e. excitability) of the cell. Such aberrant activity may be a result of e.g., but not limited to, a genetic mutation (resulting in loss-of-function or gain-of-function), loss of a regulatory mechanism, electrolyte, metabolic or hormonal imbalance, injury, loss of cells, developmental anomaly, or even due to an unknown mechanism.

Thus, according to specific embodiments, the pathological neuronal cells do not comprise a genetic mutation associated with the disease (e.g. a mutation in an ion channel e.g. SCN1A).

According to other specific embodiments, the pathological neuronal cells comprise a genetic mutation associated with the disease.

Such diseases are well known to the skilled in the art. Non-limiting examples of such diseases are listed in Table 1 hereinbelow.

According to specific embodiments, the disease is selected from the group consisting of epilepsy, Parkinson's disease, essential tremor, pain, spasticity, Achalasia, obsessive-compulsive disorder (OCD), addiction, appetite disorder, autoimmune disorder, narcolepsy and insomnia.

According to specific embodiments, the disease is epilepsy.

Epilepsy, also known as a seizure disorder, is a neurological disorder characterized by the presence of recurrent seizures. Epileptic seizures are sudden and abnormal electrical disturbances in the brain, manifested by a wide range of symptoms, any of them can be alleviated by the methods of some embodiments of the invention, including loss of consciousness, convulsions, sensory disturbances, and altered behavior. Three main types of epilepsy are known: generalized onset epilepsy, focal onset epilepsy and unknown onset epilepsy.

Diagnosing epilepsy typically involves a comprehensive evaluation by a neurologist. It is primarily based on a combination of medical history, clinical examination and various diagnostic tests e.g. a neurological exam, neuropsychological tests, blood tests, genetic testing, electroencephalogram (EEG), brain imaging tests such as computerized tomography (CT) scan, magnetic resonance imaging (MRI), Positron emission tomography (PET). It is important to note that epilepsy diagnosis is often a process of elimination, as it involves ruling out other potential causes of seizures (e.g. head injuries, toxins, tumors, and infections). To be categorized as having epilepsy, a subject must experience two or more unprovoked seizures.

Non-limiting examples of measures that can be used to assess severity of epilepsy and/or treatment efficacy include seizure frequency, seizure duration, associated symptoms, quality of life impairment.

According to specific embodiments, the disease is not epilepsy.

According to a specific embodiment, when the disease is epilepsy the first neural region (i.e. the region the polynucleotide is administered to) is not a thalamus nucleus.

According to specific embodiments, the disease is a movement disorder.

A movement disorder is a condition affecting the control and coordination of movement due to a dysfunction of the nervous system.

Such disorders are known in the art and include, for example, Parkinson's disease and essential tremor.

Parkinson's disease is manifested by a wide range of both motor and non-motor symptoms, any of them can be alleviated by the methods of some embodiments of the invention. Non-limiting examples of motor symptoms include resting tremors (tremors at rest), bradykinesia (slowness of movement), rigidity (stiffness of muscles), and postural instability (impaired balance). Non-limiting examples of non-motor symptoms may include cognitive changes, mood disturbances, sleep problems, and autonomic dysfunction.

Diagnosing Parkinson's disease is typically based on medical history, a neurological examination, and the presence of specific motor symptoms. Brain imaging may be used to rule out other conditions. Sometimes, a response to Parkinson's medication can help confirm the diagnosis.

Non-limiting examples of measure that can be used to assess severity of Parkinson's disease and/or treatment efficacy include the Unified Parkinson's Disease Rating Scale (UPDRS) (evaluates various aspects of motor symptoms, activities of daily living, and complications related to the disease), the Hoehn and Yahr staging and the Schwab and England activities of daily living scale.

Essential tremor is characterized by involuntary, rhythmic shaking movements, typically affecting the hands, but it can also involve the head, voice, or other body parts. The tremors may worsen with purposeful movement or stress and improve with rest.

Diagnosing essential tremor is typically based on clinical evaluation, medical history, and physical examination, sometimes in combination with blood tests and/or brain imaging.

Non-limiting examples of measures that can be used to assess severity of essential tremor and/or treatment efficacy include rating scales such as the Tremor Rating Scale or the Clinical Rating Scale for Tremor which evaluate the tremor's amplitude, frequency, and functional impact on daily activities.

According to specific embodiments, the disease is pain (can be persistent or recurrent).

Pain pathologies that may be treated with specific embodiments of the invention include, but not limited to, trigeminal neuralgia, migraine, occipital migraine, lower back pain and sciatica, post herpetic neuralgia, Complex Regional Pain Syndrome (CRPS), central pain and phantom pain.

Non-limiting examples of measures that can be used to assess severity of pain and/or treatment efficacy include self-reporting by the patient using scales such as visual analog scales (VAS) or numeric rating scales (NRS) to rate the intensity of pain, assessments of functional impairment, quality of life, and impact on daily activities.

According to specific embodiments, the disease is a sleep disorder.

Sleep disorders encompass a range of conditions that affect the quantity, quality, or timing of sleep.

Such diseases are known in the art and include, for example, narcolepsy and insomnia.

Narcolepsy is a neurological disorder characterized by excessive daytime sleepiness and a tendency to suddenly fall asleep at inappropriate times. Other symptoms which may be alleviated by the methods of some embodiments of the invention include cataplexy (sudden loss of muscle tone), sleep paralysis (temporary inability to move or speak while falling asleep or waking up), and vivid hallucinations during sleep-wake transitions.

Diagnosing narcolepsy involves a combination of clinical evaluation, medical history, and specialized tests, typically in combination with polysomnography (overnight sleep study) and multiple sleep latency test (MSLT) to assess sleep patterns, detect rapid eye movement (REM) sleep abnormalities, and measure daytime sleepiness.

Insomnia refers to difficulty falling asleep, staying asleep, or experiencing non-refreshing sleep despite adequate opportunity for sleep. Symptoms which may be alleviated by the methods of some embodiments of the invention include trouble initiating sleep, waking up frequently during the night, early morning awakenings, and daytime fatigue or sleepiness.

Diagnosing insomnia is primarily based on a comprehensive evaluation of sleep patterns and associated symptoms, medical history, sleep diaries, questionnaires assessing sleep quality and sleep studies.

Non-limiting examples of measures that can be used to assess severity of a sleep disorder and/or treatment efficacy include sleep logs or diaries, Epworth sleepiness scale, Insomnia Severity Index (ISI), Maintenance of Wakefulness Test (MWT).

According to specific embodiments, the disease is a psychiatric disorder.

Such disorders are known in the art and include, for example, obsessive-compulsive disorder (OCD) and addiction.

Diagnosis of OCD and addiction may be affected by the specific criteria outlined in the DSM-5.

A non-limiting example of a measure that can be used to assess severity of OCD and/or treatment efficacy include the Yale-Brown Obsessive Compulsive Scale (Y-BOCS).

Non-limiting examples of measures that can be used to assess severity of addiction and/or treatment efficacy include the Addiction Severity Index (ASI) or the Substance Abuse Subtle Screening Inventory (SASSI).

According to specific embodiments, the disease is a neuromuscular disease.

According to specific embodiments, the disease is a neuromuscular disorder.

Neuromuscular disorders encompass a range of conditions characterized by an abnormal increase in muscle tone or stiffness of muscle groups, leading to dysfunction of said muscles. The underlying pathology could be secondary to a CNS lesion (e.g. Stroke, Cerebral Palsy, spinal cord injury, etc.) or due to local muscle pathologies (e.g. loss of distal inhibitory neurons in the Lower Esophageal Sphincter, which leads to increased tone and incomplete relaxation of the sphincter—causes Achalasia).

Such diseases are known in the art and include, for example, but not limited to, spasticity, achalasia, urinary retention.

Spasticity is a feature of pathological skeletal muscle performance with a combination of paralysis, increased tendon reflex activity, and increased tone. Clinically, spasticity results from the loss of inhibition of motor neurons, causing excessive muscle contraction. This ultimately leads to hyperreflexia, an exaggerated deep tendon reflex, stiffness and paralysis of affected muscles. It is typically caused by a lesion in the brain or spinal cord (Upper Motor Neuron lesion such as stroke, cerebral palsy, spinal cord injury, multiple sclerosis, etc.) leading to a decrease of inhibition and subsequent increased excitability of muscles.

Diagnosing spasticity is primarily assessed by physical examination, medical history, and imaging techniques like CT or MRI.

Non-limiting examples of measures that can be used to assess severity of spasticity and/or treatment efficacy include scales such as the King's hypertonicity scale, the Tardieu scale, and the modified Ashworth scale.

Achalasia (or Esophageal Achalasia) is characterized by a failure of smooth muscle fibers of the Lower Esophageal Sphincter to relax. The Lower Esophageal Sphincter (LES) is a muscle located between the stomach and the esophagus that opens when food comes in and closes in order to prevent food and stomach acid from traveling up the esophagus. Several etiologies exist, but the most common ones involve the failure of distal inhibitory neurons in the LES, leading to increased muscle tone and failure to relax. Symptoms include dysphagia (difficulty swallowing), regurgitation of undigested food, foul breath odor, chest pain and weight loss.

Diagnosing Achalasia involves physical examination, medical history and more prominently by Barium swallowing, esophageal manometry tests, and endoscopy.

Urinary retention is characterized by an inability to urinate and abdominal pain due to a distended bladder. There are several etiologies that might lead to urinary retention, some of which are caused due to neurological conditions (e.g. spinal cord injury, diabetes, stroke, trauma and heavy metal poisoning. A subset of these etiologies leads to sphincter increased tone or over-activation.

Evaluating bladder function and the ability to empty it typically includes Ultrasound examination and assessment of residual urine within the bladder post-urination.

According to specific embodiments, the disease is an autoimmune disease.

According to specific embodiments, the autoimmune disease is TNFα-mediated.

According to specific embodiments, the autoimmune disease is a disease known to be ameliorated by treatment with a TNF inhibitor.

Such diseases are known in the art and include, for example, inflammatory bowel diseases like ulcerative colitis (UC) or Crohn's disease, rheumatoid arthritis, ankylosing spondylitis, and psoriasis.

Ulcerative colitis (UC), for example, is a chronic inflammatory bowel disease (IBD) that primarily affects the colon (large intestine) and rectum. It is characterized by inflammation and ulcers in the lining of the colon. Symptoms which may be alleviated by the methods of some embodiments of the invention include diarrhea, abdominal pain and cramping, rectal bleeding, tenesmus, fatigue, weight loss, loss of appetite, anemia.

Diagnosing UC is typically based on medical history, blood tests, stool sample analysis, colonoscopy and imaging tests (x-rays, CT, MRI).

Non-limiting examples of measures that can be used to assess severity of UC and/or treatment efficacy include clinical symptoms (the frequency and severity of diarrhea, rectal bleeding, abdominal pain, and urgency to have bowel movements), endoscopic findings (the appearance of the colon's lining as observed during a colonoscopy), laboratory markers [e.g. inflammatory markers such as C-reactive protein (CRP) and erythrocyte sedimentation rate (ESR)], various scoring systems, such as the Mayo score or the Simple Clinical Colitis Activity Index (SCCAI).

According to specific embodiments, the disease is a neuroendocrine disease.

A neuroendocrine disease is manifested by unregulated hormone production and/or secretion which leads to pathological hormonal imbalance that might culminate in systemic disease.

Such diseases are known in the art and include, for example eating disorders characterized by appetite dysregulation (e.g. anorexia nervosa, binge eating disorder, bulimia nervosa) or satiety disorders leading to obesity (e.g. Prader Willi Syndrome, and others), as well as Cushing's disease (characterized by excessive secretion of ACTH).

According to specific embodiments, the hormone is not a stress hormone.

According to specific embodiments, the hormone is not adrenocorticotropic hormone (ACTH) or corticosterone.

According to specific embodiments, the disease is not an eating disorder.

According to specific embodiments, the pathological neuronal cell associated with the disease is not an agouti-related peptide (AgRP)-expressing neuron (e.g. fasting-activated hypothalamic AgRP-expressing neuron).

Non-limiting examples of measures that can be used to assess severity of an appetite disorder or obesity and/or treatment efficacy include assessments of body weight, body mass index (BMI), psychological well-being, and associated physical complications.

The methods disclosed herein include administering into a first neural region of the subject a polynucleotide encoding a bistable type II opsin attached to a heterologous ER export signal and/or membrane trafficking signal. Such polynucleotides are disclosed for example in International Patent Application Publication No. WO2020/188572, the contents of which are fully incorporated herein by reference, and are further described hereinbelow.

The first neural region comprises cell bodies of the pathological neuronal cells that transmit the aberrant signal associated with the disease. Such neural regions are known in the art and disclosed for example in The Netter's Atlas of NeuroScience, The Neurosurgical Atlas, Human Connectome models and the like, and several specific examples are provided in Table 1 hereinbelow.

Typically, to allow direct administration to the specific cell bodies of interest of a subject, the polynucleotide is administered in a local manner. However, systemic administration is also contemplated, by specific embodiments, in a manner enabling specific expression of the bistable type II opsin by neuronal cell bodies of the first neural region. Non-limiting examples of such systemic administration include, systemic injection of AAVs comprising a polynucleotide encoding the bistable type II opsin following Focused Ultrasound treatment to the targeted brain region [see e.g. Rikke Hahn Kofoed, et al. (2022) Journal of Controlled Release, 351: 667-680], and the use of BBB-crossing vectors/capsids (see e.g. Chuapoco M R, et al. Res Sq 2023 Jan. 13:rs.3.rs-1370972. Doi: 10.21203/rs.3.rs-1370972/vl; update in: Nat Nanotechnol. 2023 Jul. 10 doi: 10.1038/s41565-023-01419-x).

According to specific embodiments, the polynucleotide is administered intrathecally (IT), Intracerebrally (ICM), intracerebroventricularly (ICV) or intranasally (IN).

According to specific embodiments, the administering or administration is by a stereotactic injection.

As used herein, the term “stereotactic injection” refers to a technique for delivering a substance or medication to a precise and predetermined target site within the brain. It involves the use of three-dimensional coordinates to guide the placement of a needle or catheter with high accuracy. According to specific embodiments, the stereotactic injection is MRI guided. The stereotactic injection allows for targeted delivery of the substance to the desired location, minimizing damage to surrounding tissues and optimizing therapeutic efficacy (e.g. minimizing off-target effects and allowing the use of smaller titers and dosages, making the treatment safer and cheaper).

A Type II opsin is a G-coupled protein receptor (GPCR) which is made light-sensitive with an attached retinal chromophore molecule which acts as a light sensor. Most type II opsins bind 11-cis retinal as a chromophore to form a photosensitive pigment (opsin-based pigment). The isomerization of the chromophore (e.g. 11-cis to all-trans) in an opsin-based pigment upon light absorption triggers G protein activation.

According to specific embodiments, the Type II opsin activates Gᵢ-type and Gₒ-type G protein in a light dependent manner.

According to specific embodiments, the Type II opsin activates G_{z}-type G protein in a light dependent manner.

Type II opsins do not comprise an ion channel or a proton/ion pump.

As used herein, the phrase “bistable type II opsin” refers to a type II opsin which remains bound to the retinal chromophore following illumination (i.e. does not undergo bleaching).

Hence, a bistable type II opsin displays prolonged signal transduction following a single illumination pulse. Typically, the bistable type II opsin reverts to an original dark state through thermal relaxation after minutes in the dark or by illumination with light at a different wavelength.

Methods of determining bistability of the opsin are well known in the art and include spectroscopic measurements.

According to specific embodiments, the bistable type II opsin is a naturally occurring bistable type II opsin. Such naturally occurring bistable type II opsins are known in the art and include, but are not limited to OPN3 (e.g. MosOpn3), OPN4, OPN5, parapinopsin (e.g. LcPP, zPP1, pPP2, zPP2/DrPP2, pPP2/TrPP2), PdCO (e.g. PdCO2), TMT (e.g. PufTMT, medakaTMT1A), peropsin.

According to specific embodiments, the bistable type II opsin is selected from the group consisting of OPN3, OPN4, OPN5, parapinopsin, zPP2, pPP2, PdCO, TMT and peropsin According to specific embodiments, the bistable type II opsin is selected from the group consisting of OPN3, OPN4, OPN5, parapinopsin, PdCO and peropsin.

According to specific embodiments, the bistable type II opsin is selected from the group consisting of OPN3, parapinopsin, PdCO and TMT.

According to specific embodiments, the bistable type II opsin is selected from the group consisting of OPN3, parapinopsin and PdCO.

Any of the bistable type II opsins disclosed herein also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the sequences of the wild type opsins disclosed herein (as also exemplified by specific accession numbers and amino acid sequences); or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the opsin may comprise conservative and non-conservative amino acid substitutions.

According to specific embodiments, the bistable type II opsin activates G_{i/o} signaling in a cell expressing same following exposure to light in a wavelength that activates it, as determined by e.g. GsX assay (Ballister, et al., 2018); or the ability to evoke G protein-coupled inwardly-rectifying potassium channel-mediated (GIRK) currents in neurons expressing a GIRK2-1 channel, as described in details in the Examples section which follows.

According to specific embodiments, the bistable type II opsin activates Gz signaling in a cell expressing same following exposure to light in a wavelength that activates it, as determined by e.g. GsX assay (Ballister, et al., 2018).

According to specific embodiments, the bistable type II opsin is OPN3.

As used herein, the term “OPN3” refers to the vertebrate Opsin-3, also known as encephalopsin or panopsin, and any homolog thereof.

According to specific embodiments, the OPN3 is the mosquito (Anopheles stephensi) OPN3 (MosOpn3), such as provided in the following Accession Number: BAN05625.

According to specific embodiments, the MosOpn3 amino acid sequence comprises SEQ ID NO: 8.

According to specific embodiments, the MosOpn3 amino acid sequence consists of SEQ ID NO: 8.

According to specific embodiments, the MosOpn3 amino acid sequence is the amino acid sequence described in Koyanagi et al. (Proc Natl Acad Sci USA. 2013 Mar. 26; 110(13): 4998-5003), the content of which are fully incorporated herein by reference.

According to other specific embodiments, the MosOpn3 amino acid sequence is not the amino acid sequence described in Koyanagi et al. (Proc Natl Acad Sci USA. 2013 Mar. 26; 110(13): 4998-5003).

According to specific embodiments, the MosOpn3 amino acid sequence comprises SEQ ID NO: 9.

According to specific embodiments, the MosOpn3 amino acid sequence consists of SEQ ID NO: 9.

According to specific embodiments, the MosOpn3 amino acid sequence does not consist of SEQ ID NO: 9.

The term “MosOpn3” also encompasses functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 8; or at least 70%, at least 75%, at least 80%, at least 81%, at 20 least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is TMT, also known as Teleost multiple tissue.

According to other specific embodiments, the bistable type II opsin is not TMT.

According to specific embodiments, the TMT is the pufferfish teleost multiple tissue opsin (PufTMT) such as provided in the following Accession Number: AAM90677.

According to other specific embodiments, the bistable opsin II is not the pufferfish teleost multiple tissue opsin (PufTMT).

According to specific embodiments, the PufTMT amino acid sequence comprises SEQ ID NO: 10.

According to specific embodiments, the PufTMT amino acid sequence consists of SEQ ID NO: 10.

According to specific embodiments, the TMT is TMT1A such as the medaka teleost multiple tissue opsin 1A (medakaTMT1A) such as provided in the following Accession Number: AGK24990.

According to specific embodiments, the medakaTMT1A amino acid sequence comprises SEQ ID NO: 33.

According to specific embodiments, the medakaTMT1A amino acid sequence consists of SEQ ID NO: 33.

According to specific embodiments, the PufTMT or medakaTMT1A amino acid sequence is the amino acid sequence described in Sakai K. et al. [PLoS ONE (2015) 10(10): e0141238], the content of which are fully incorporated herein by reference.

The terms “PufTMT”, “medakaTMT1A” also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, 15 at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 10, 33, respectively; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at 20 least 93%, at least 94%, at least 95%, at least 96%, at least 97%, at least 98%, at least 99% or 100% identical to the polynucleotide sequence encoding same.

According to specific embodiments, the bistable type II opsin is parapinopsin. Non-limiting examples of parapinopsins include Lethenteron camtschaticum parapinopsin (LcPP), zebrafish parapinopsin 1 (zPP1), zebrafish parapinopsin 2 [zPP2, also known as Danio rerio parapinopsin2 (drPP2)], pufferfish parapinopsin (pPP2, also known as TrPP2).

According to specific embodiments, the parapinopsin is the Lethenteron camtschaticum (Lamprey) parapinopsin (LcPP) such as provided in the following Accession Number: BAD13381.

According to specific embodiments, the LcPP amino acid sequence comprises SEQ ID NO: 29.

According to specific embodiments, the LcPP amino acid sequence consists of SEQ ID NO: 29.

According to specific embodiments, the LcPP amino acid sequence is the amino acid sequence described in Eickelbeck et al. [ChemBioChem (2020) 21: 612-617], the content of which are fully incorporated herein by reference.

According to specific embodiments, the parapinopsin is the zebra fish parapinopsin 1 (zPP1) such as provided in the following Accession Number: AB626966.

According to specific embodiments, the zPP1 amino acid sequence comprises SEQ ID NO: 37.

According to specific embodiments, the zPP1 amino acid sequence consists of SEQ ID NO: 37.

According to specific embodiments, the zPP1 amino acid sequence is the amino acid sequence described in Kawano-Yamashita E. et al. [PLoS ONE (2015) 10(10): e0141280], the content of which are fully incorporated herein by reference.

According to specific embodiments, the parapinopsin is the pufferfish parapinopsin (pPP2) such as provided in the following Accession Number: AB626965.

According to specific embodiments, the pPP2 amino acid sequence comprises SEQ ID NO: 41.

According to specific embodiments, the pPP2 amino acid sequence consists of SEQ ID NO: 41.

The terms “LcPP”, “zPP1”, “pPP2” also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 29, 37, 41, respectively; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is PdCO, also known as Platynereis dumerilii ciliary opsin.

According to specific embodiments, the PdCO is the PdCO2 such as provided in the following Accession Number: AY692353.

According to specific embodiments, the PdCO2 amino acid sequence comprises SEQ ID NO: 25.

According to specific embodiments, the PdCO2 amino acid sequence consists of SEQ ID NO: 25.

According to specific embodiments, the PdCO amino acid sequence is the amino acid sequence described in Tsukamoto et al. [J. Biol. Chem. (2017) doi: 10.1074/jbc.M117.793539], the content of which are fully incorporated herein by reference.

The term “PdCO2” also encompasses functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 25; or at least 70%, at least 75%, at least 80%, at least 81%, at 15 least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the bistable type II opsin is selected from the group consisting of MosOpn3, LcPP, zPP1, pPP2, PdCO2, PufTMT and medakaTMT1A.

According to specific embodiments, the bistable type II opsin is selected from the group consisting of MosOpn3, LcPP, zPP1, pPP2, PdCO2 and medakaTMT1A.

The polynucleotides disclosed herein encode a bistable type II opsin attached to an ER export signal and/or membrane trafficking signal heterologous to the bistable type II opsin.

As used herein, the term “heterologous” refers to a sequence which is not native to the bistable type II opsin at least in localization or is completely absent from the native sequence of the polypeptide. The heterologous moiety forms a chimeric or a fusion polypeptide.

According to specific embodiments, the heterologous ER export signal and/or membrane trafficking signal is located C-terminally to the bistable type II opsin.

According to specific embodiments, the heterologous ER export signal and/or membrane trafficking signal enables trafficking to axon and/or dendrite terminals. To render explicit, according to specific embodiments, the ER export signal and/or membrane trafficking signal enables membrane expression or presentation of the bistable type II opsin. Methods of determining trafficking to axon and dendrite terminals are well known in the art and include for example immunostaining and fluorescence microscopy. Alternatively or additionally, determining may be performed by electrophysiological and behavioral methods, such as disclosed for example in Mahn et al. (2021) Neuron 109: 1621-1635.

ER export signals are known in the art, and disclosed e.g. in Stockklausner et al., FEBS Lett.; 493 (2-3):129-133 March, 2001; Ma et al., Science Vol. 291. no. 5502:316-319, 2001); Paulhe et al., J. Biol. Chem., Vol. 279, Issue 53, 55545-55555, Dec. 31, 2004); Farhan et al., J.

Cell Sci. 121:753-761, Feb. 19, 2008; the contents of each are incorporated herein by reference in their entirety.

According to specific embodiments, the ER export signal is of a protein expressed in neuronal cells.

According to specific embodiments, the ER export signal is of a protein expressed in the axons or the presynaptic terminals of neuronal cells.

Non-limiting examples ER export signals can be the signals of the inward rectifier potassium channel Kir2.1, NgCAM, VAMP2, Neurexin, Synapsin, Synaptophysin, Synaptotagmin, SynCAM, Piccolo or Basoon.

According to specific embodiments, the ER signal is of the inward rectifier potassium channel Kir2.1.

Non-limiting examples of amino acid sequence of ER export signals that can be used with specific embodiments of the invention include, FXYENE (SEQ ID NO: 11, where X is any amino acid), e.g. FCYENEV (SEQ ID NO: 2); VXXSL (where X is any amino acid), e.g. VKESL (SEQ ID NO: 13); VLGSL (SEQ ID NO: 14); NANSFCYENEVALTSK (SEQ ID NO: 15); C-terminal valine residue; and VMI.

According to specific embodiments, the amino acid sequence of the ER export signal is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to an amino acid sequence selected from the group consisting of SEQ ID NO: 2, 13 and 14, each possibility represents a separate embodiments of the invention.

According to specific embodiments, the amino acid sequence of the ER export signal comprises SEQ ID NO: 2.

According to specific embodiments, the amino acid sequence of the ER export signal consists of SEQ ID NO: 2.

According to specific embodiments, the ER export signal amino acid sequence is 5-25 amino acids in length, e.g. 5-10, 10-15, 15-20, 20-25 amino acids in length.

Membrane trafficking signals are known in the art, and include, but are not limited to membrane trafficking signals of a protein expressed on the membranes of neuronal cells.

According to specific embodiments, the membrane trafficking signal is of a protein expressed in neuronal cells.

According to specific embodiments, the membrane trafficking signal is of a protein expressed in the axons or the presynaptic terminals of neuronal cells.

Non-limiting examples of membrane trafficking signals can be the signals of the inward rectifier potassium channel Kir2.1, the hChR2, the neuronal nicotinic acetylcholine receptor, NgCAM, VAMP2, Neurexin, Synapsin, Synaptophysin, Synaptotagmin, SynCAM, Piccolo or Basoon.

According to specific embodiments, the trafficking signal is of a Kir2.1 polypeptide.

Amino acid sequence of trafficking sequences that are suitable for use with specific embodiments include, but are not limited to KSRITSEGEYIPLDQIDINV (SEQ ID NO: 1), MDYGGALSAVGRELLFVTNPVVVNGS (SEQ ID NO: 16), MAGHSNSMALFSFSLLWLCSGVLGTEF (SEQ ID NO: 17), MGLRALMLWLLAAAGLVRESLQG (SEQ ID NO: 18), MRGTPLLLVVSLFSLLQD (SEQ ID NO: 19).

According to specific embodiments, the amino acid sequence of membrane trafficking signal is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to an amino acid sequence selected from the group consisting of SEQ ID NO: 1, 16, 17, 18 and 19, each possibility represents a separate embodiments of the invention.

According to specific embodiments, the amino acid sequence of the membrane trafficking signal comprises SEQ ID NO: 1.

According to specific embodiments, the amino acid sequence of the membrane trafficking signal consisting of SEQ ID NO: 1.

According to specific embodiments, the membrane trafficking signal amino acid sequence is 10-50 amino acids in length, e.g. 10-20, 20-30, 30-40, 40-50 amino acids in length.

As used herein the term “polynucleotide”, refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule.

Accordingly, silent variations of a polynucleotide which encodes a polypeptide is implicit in a described sequence with respect to the expression product.

According to specific embodiments, the polynucleotides or nucleic acid sequences disclosed herein are codon optimized to heterologous (e.g. mammalian) expression.

Methods of codon optimization are known in the art and disclosed e.g. in Grote et al. (Nucleic Acid Res. Nucleic Acids Res. (2005) Jul. 1; 33(Web Server issue): W526-W531) and include e.g. mouse codon usage optimized or human codon usage optimized versions.

Hence, according to specific embodiments, the nucleic acid sequence of the MosOpn3 comprises SEQ ID NO: 20.

According to specific embodiments, the nucleic acid sequence of the MosOpn3 consists of SEQ ID NO: 20.

According to specific embodiments, the nucleic acid sequence of the MosOpn3 comprises SEQ ID NO: 21.

According to specific embodiments, the nucleic acid sequence of the MosOpn3 consists of SEQ ID NO: 21.

According to specific embodiments, the nucleic acid sequence of the PufTMT comprises SEQ ID NO: 22.

According to specific embodiments, the nucleic acid sequence of the PufTMT consists of SEQ ID NO: 22.

According to specific embodiments, the nucleic acid sequence of the PufTMT comprises SEQ ID NO: 23.

According to specific embodiments, the nucleic acid sequence of the PufTMT consists of SEQ ID NO: 23.

According to specific embodiments, the nucleic acid sequence of the medakaTMT1A comprises SEQ ID NO: 34.

According to specific embodiments, the nucleic acid sequence of the medakaTMT1A consists of SEQ ID NO: 34.

According to specific embodiments, the nucleic acid sequence of the LcPP comprises SEQ ID NO: 30.

According to specific embodiments, the nucleic acid sequence of the LcPP consists of SEQ ID NO: 30.

According to specific embodiments, the nucleic acid sequence of the zPP1 comprises SEQ ID NO: 38.

According to specific embodiments, the nucleic acid sequence of the zPP1 consists of SEQ ID NO: 38.

According to specific embodiments, the nucleic acid sequence of the pPP2 comprises SEQ ID NO: 42.

According to specific embodiments, the nucleic acid sequence of the pPP2 consists of SEQ ID NO: 42.

According to specific embodiments, the nucleic acid sequence of the PdCO2 comprises SEQ ID NO: 26.

According to specific embodiments, the nucleic acid sequence of the PdCO2 consists of SEQ ID NO: 26.

To express an exogenous polypeptide in mammalian cells, a polynucleotide encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

Hence, according to specific embodiments, the polynucleotide is comprised a in a nucleic acid construct comprising the polynucleotide and a regulatory element for directing expression of the polynucleotide in a cell (e.g. promoter).

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Thus, according to specific embodiments, promoter is a neuron specific promoter. Non-limiting examples of neuron-specific promoters include the neurofilament promoter [Byrne et al. (1989) Proc. Natl. Acad. Sci. USA 86:5473-5477; or GenBank HUMNFL, L04147], neuron-specific enolase (NSE) promoter (see, e.g., EMBL HSENO2, X51956; see also, e.g., U.S. Pat. Nos. 6,649,811, 5,387,742); aromatic amino acid decarboxylase (AADC) promoter; synapsin promoter (see, e.g., GenBank HUMSYNIB, M55301); thy-1 promoter (see, e.g., Chen et al. (1987) Cell 51:7-19; and Llewellyn et al. (2010) Nat. Med. 16:1161); serotonin receptor promoter (see, e.g., GenBank S62283); tyrosine hydroxylase promoter (TH) (see, e.g., Nucl. Acids. Res. 15:2363-2384 (1987) and Neuron 6:583-594 (1991)); GnRH promoter (see, e.g., Radovick et al., Proc. Natl. Acad. Sci. USA 88:3402-3406 (1991)); L7 promoter (see, e.g., Oberdick et al., Science 248:223-226 (1990)); DNMT promoter (see, e.g., Bartge et al., Proc. Natl. Acad. Sci. USA 85:3648-3652 (1988)); enkephalin promoter (see, e.g., Comb et al., EMBO J. 17:3793-3805 (1988)); a myelin basic protein (MBP) promoter; CMV enhancer/platelet-derived growth factor-β promoter (see, e.g., Liu et al. (2004) Gene Therapy 11:52-60); motor neuron-specific gene Hb9 promoter (see, e.g., U.S. Pat. No. 7,632,679; and Lee et al. (2004) Development 131:3295-3306); alpha subunit of Ca⁽²⁺⁾-calmodulin-dependent protein kinase II (CaMKIIα) promoter (see, e.g., Mayford et al. (1996) Proc. Natl. Acad. Sci. USA 93:13250) and the Orexin promoter (described in e.g. Moriguchi T et al. (2002) J Biol Chem. 277(19): 16985-92).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long terminal repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference. Enhancers specific for distinct neuronal cell types that can be included in AAV expression vectors to gain specificity without a Cre-driver line have also been described in the arts and described e.g. in Hrvatin et al. (doi: www(dot)//doi(dot)org/10.1101/570895), which is incorporated herein by reference. Cell-type specific enhancers, such as described in e.g. Juittner e al. [Nature Neuroscience volume 22, pages1345-1356 (2019)] or Dimidschstein et al. (Nature Neuroscience volume 19, pages1743-1749 (2016)], the contents of which are incorporated herein by reference, for expression in inhibitory interneurons.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the polynucleotide.

Thus, according to specific embodiments, the polynucleotide or vector comprises a genomic integration sequence, such that upon administration, the polynucleotide is integrated in the genome of the infected or transfected cell.

According to other specific embodiments, the polynucleotide or vector does not comprise a genomic integration sequence, such that upon administration the polynucleotide does not integrate with the genome of the infected or transfected cell. In such cases, specific embodiments suggest the formation of an episome.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of the polypeptides since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Any of the components comprised in the polynucleotide as described herein may be linked to each other directly or via a linker, each possibility represents a separate embodiment of the present invention.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference.

According to specific embodiments, the amino acid sequence of the linker is selected from the group consisting of PRARDP (SEQ ID NO: 4), (Gly)ₙ (where n indicates vari-able copy numbers), (GₙSₙ)l (where n indicates variable copy numbers), ((GₙSₙ)ₙPₙ)ₙ (where n indicates variable copy numbers) and (EAAAK)ₙ (where n indicates variable copy numbers, SEQ ID NO: 24).

According to specific embodiments, the polynucleotide may comprise or encode epitope tags, fluorescent proteins, cleavable linker peptides, a cell penetrating moiety, targeting moieties and the like.

According to specific embodiments, the polynucleotide encodes an amino acid sequence for directing the bistable type II opsin to a specific membrane location e.g. the axon or the presynaptic terminal.

Various methods can be used to introduce the polynucleotide or expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. Introduction of nucleic acids by viral infection offers several advantages over other methods such as lipofection and electroporation, since higher transfection efficiency can be obtained due to the infectious nature of viruses. Thus, according to specific embodiments, the polynucleotide is packed in a viral vector (e.g. AAV). Other vectors can be used that are non-viral, such as cationic lipids, polylysine, and dendrimers.

The polynucleotides and nucleic acid constructs of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polynucleotides, nucleic acid constructs and polypeptides encoded therefrom accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen. Thus, for example, for injection, the active ingredients of the pharmaceutical composition may be formulated in aqueous solutions, preferably in physiologically compatible buffers such as Hank's solution, Ringer's solution, or physiological salt buffer. Alternatively, the active ingredient may be in powder form for constitution with a suitable vehicle, e.g., sterile, pyrogen-free water based solution, before use.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

Non-limiting examples of animal models that can be used with specific embodiments of the invention are described in the Examples section which follows and include for example a genetic Slc6al model or the SCN1a (Dravet) genetic model for generalized onset epilepsy and the Kainate model for focal onset epilepsy and [see www(dot)informatics.jax(dot)org/marker/MGI:95627; Lindquist et al. bioRxiv preprint doi: www(dot)doi(dot)org/10.1101/2021.12.17.473036; Lévesque M, et al. (20123) Neurosci Biobehav Rev. 37(10 Pt 2):2887-99; and Twele F et al. (2017) Epilepsia Open. 2(2):180-187], a 6-OHDA Parkinson's model [see e.g. Masini D, et al. (2021) Biomedicines. 9(6): 598], acute headaches and migraines rodent model [e.g. Dural Capsaicin model, a chemical model of acute headache described in e.g. Huang D et al. (2016) Pain 157(8): 1744-1760, Von Frey microfilaments and thermal allodynia using acetone evaporation test and Hargreaves' test, a mechanical model of acute pain described in e.g. Vuralli, D. et al. (2019) J Headache Pain 20, 11. www(dot)doi(dot)org/10.1186/s10194-019-0963-6)], a peripheral pain model using a hot plate setting [see e.g. Dale J. Langford, Jeffrey S. Mogil, in Anesthesia and Analgesia in Laboratory Animals (Second Edition), 2008]. Closed- and open-loop approaches are described for example in Paz et al. Nat Neurosci (2013)16(1): 64-70; and Salanova V. et al.; SANTE Study Group. (2021) Epilepsia. 62(6): 1306-1317, the contents of which are fully incorporated herein by reference.

The doses determined in the rodent animal model can be converted for the treatment of other species such as human and other animals diagnosed with the disease, using conversion Tables known to those skilled in the art.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration.

Depending on the severity and responsiveness of the condition to be treated, dosing can be of a single or a plurality of administrations, with course of treatment lasting from several days to several weeks or until cure is effected or diminution of the disease state is achieved, as further described hereinbelow.

The amount of a composition to be administered will, of course, be dependent on the subject being treated, the severity of the affliction, the manner of administration, the judgment of the prescribing physician or neurologist, etc. According to specific embodiments, the amount to be administered depends on the judgment of the neurosurgeon, in accordance with electrophysiological measurements (e.g. EEG) or imaging e.g. MRI.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Following administration of the polynucleotide, the methods disclosed herein comprise exposing a second neural region of the subject to light in a wavelength that activates the bistable type II opsin.

Such a wavelength typically depends on the type of the bistable type II opsin. Determining the suitable wavelength is well within the capabilities of the skilled in the art. According to specific embodiments, the light may range from ultraviolet down to near-infrared light. According to specific embodiments, the light is an ultraviolet, blue, green, yellow or red light.

According to specific embodiments, the wavelength is 350-670 nm.

According to specific embodiments, the wavelength is 450-650 nm.

According to specific embodiments, the wavelength is 450-490 nm.

According to specific embodiments, the wavelength is about 470 nm.

According to specific embodiments, the wavelength is 540-580 nm.

According to specific embodiments, the wavelength is about 560 nm.

According to specific embodiments, the wavelength is 610-650 nm.

According to specific embodiments, the wavelength is about 630 nm.

According to specific embodiments, the wavelength is 650-700 nm.

According to specific embodiments, the wavelength is about 680 nm.

According to specific embodiments, exposing to light is effected by light pulses that can have a duration for any of at least 1 millisecond (ms), at least 5 ms, at least 10 ms, at least 50 ms, at least 100 ms, at least 500 ms, at least 1 sec, at least 5 sec, at least 10 sec, at least 20 sec, at least 30 sec, at least 40 sec.

According to specific embodiments, exposing to light is effected by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 sec, about 1.25 sec, about 1.5 sec, about 2 sec, about 5 sec, about 10 sec, about 20 sec, about 30 sec, about 40 sec.

According to specific embodiments, exposing to light is effected by a light pulse having a duration of 0.5-30 seconds.

The second neural region comprises axon or dendrite terminals of the pathologic neuronal cells associated with a symptom of the disease. Further, the second neural region is distinct from the first neural region disclosed herein. To reiterate, the second neural region is linked to the first neural region by projections/axons or dendrites reaching it from the first neural region.

According to specific embodiments, the second region comprises axon terminals.

According to specific embodiments, the axon terminals are presynaptic terminals.

According to specific embodiments, the second region comprises dendrite terminals.

The dendrite terminals encompassed by specific embodiments of the invention release neuropeptides or neurotransmitters.

As used herein, the phrase “distinct neural region” indicates that illumination is performed such that the second region gets effective illumination to activate the bistable type II opsin expressed therein, while the first region does not get effective illumination to activate the bistable type II opsin expressed in the first region.

Projections of cell bodies and their specific axon/dendrite terminals associated with diseases are well known in the art and documented, for example, in mechanisms of seizure generalization in generalized epilepsy, in which thalamic neurons' projections to various cortical areas play crucial role in seizure propagation [Chen, Y et al. (2021) Human Brain Mapping, 42 17), 5648-5664]; projections within the Basal Ganglia explaining the underlying mechanism leading to Parkinson's disease [Jae-Hyuk Shim, Hyeon-Man Baek, (2022) Neuroscience, 483: 32-39]; or the connectivity and neuronal projections to and from the Nucleus Accumbens in Addiction [Yang Xia, et al. (2021) Frontiers in Psychiatry, 12, doi: 10.3389/fpsyt.2021.609458]. Hence, determination of the second neural regions for each disease is well within the capabilities of those skilled in the art, and depends on the specific first region injected and the specific disease. Non-limiting examples of the combination of the disease, the first neural region and the second neural region that can be used with specific embodiments of the invention are provided in Table 1 hereinbelow.

Methods and apparatuses for exposing such neural regions to such wavelengths are known in the art and include for example the Clinatec device for PhotoBioModulation therapy (PBMT) disclosed in www(dot)nature(dot)com/articles/d41586-023-00079-0 and the Blackrock Neurotech's Opto-array described in www(dot)nature(dot)com/articles/s41592-021-01238-9e. According to specific embodiments, the device is implanted into the subject. Thus, for example, the device may be a skull-mounted device, an intracranial device, a device implanted along the spine and the like.

According to specific embodiments, exposing is effected using a skull-mounted device. For example, the device may comprise a uLED array aimed to be placed intracranially or extracranially/subdermally/epidurally, and is conjoined with depth/surface/epidural/subdermal/external electrodes aimed at recording electrophysiological neural activity from various brain locations.

According to other specific embodiments, the device is external to the subject. Non-limiting examples of such devices include a cell phone, a flashlight and the like.

According to specific embodiments, the device allows electrical recording and optical stimulation in parallel—in closed-loop and/or open-loop, and/or patient- or caretaker-controlled manners.

Care should be taken to expose the neural region to light following an amount of time allowing expression of the polypeptide encoded by the polynucleotide or the nucleic acid construct. Hence, according to specific embodiments, exposing is effected following an amount of time allowing expression of the bistable type II opsin in the pathologic neuronal cells. According to specific embodiments, exposing is effected following an amount of time allowing membrane expression or presentation of the bistable type II opsin. According to specific embodiments, exposing is effected following an amount of time allowing expression of the bistable type II opsin in the axon and/or dendrite terminals of the pathologic neuronal cells.

Thus, according to specific embodiments, the exposing is effected at least 1 week, at least 2 weeks, at least 3, weeks, at least 4 weeks, at least 5 weeks, at least 6 weeks, at least 7 weeks, at least 8 weeks, at least 9 weeks, at least 10 weeks following the administering.

According to a specific embodiment, exposing is effected at least 6 weeks following said administering.

According to a specific embodiment, exposing is effected at least 8 weeks following said administering.

Exposing to light may be effected independent or dependent of detection of an acute symptom of the disease.

According to specific embodiments, exposing to light is effected upon detection of an acute symptom of the disease (may be also referred to as the “closed-loop approach”). Such detection may be for example by electrical means (electrophysiological recording, e.g. by EEG, ECOG and/or SEEG (according to amplitude, spiking, wave pattern) using electrodes (e.g. external, epidural, subdural, or depth electrodes) or by detection of symptoms such as disclosed hereinabove; and can be effected automatically by a device or by the subject or caretaker.

Thus, for example, the skull-mounted device may allow electrophysiological(EEG, ECOG and/or SEEG) recording; such that upon detection of an acute symptom of the disease (e.g. using an algorithm identifying initiation of a symptom) a light pulse(s) will be transmitted.

Alternatively, or additionally, the subject may actively start illumination upon detecting an onset of a symptom of the disease.

According to other specific embodiments, exposing to light comprises repeated illumination independent (may be also referred to as the “open-loop approach”) of detection of an acute symptom of the disease.

Under such a scenario, illumination is delivered in a preset cycle and not directly in response to an initiation of a symptom and may be either in a continuous (or chronic) or alternating manner.

As the bistable type II opsins of some embodiments of the invention remain active for about 5 minutes following an illumination pulse; a light pulse every 10 seconds-5 minutes will result in a continuous active opsin and a light pulse every more than 5 minutes (e.g. every 10-30 minutes) will result in alternating activation of the opsin.

The preset cycle may be controlled by a pre-set program and/or by the subject in an active manner (e.g. before performing an activity such as driving and the like).

It should be noted that according to specific embodiments, it is possible to reverse activation of the expressed bistable II opsin using light in a wavelength different than the one that activates it, enabling easier regulation of the amount and duration of activation. Thus, according to specific embodiments, the method comprises exposing the second neural region of the subject to light in a wavelength that inhibits activation of the polypeptide.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

TABLE 1

| | | | Illumination | Mechanism Of | |
|---|---|---|---|---|---|
| Disorder | Indication | Injection target | target | Action | Desired effect |
| Seizure | Focal | Focus site | Areas having | Inhibition of | a. Seizure |
| disorders | seizures | | projections from | eOPN3 | frequency |
| | 1. TLE or | | focus site, such | expressing | reduction |
| | frontal | | as thalamic | terminals of | b. Seizure |
| | foci | | nuclei, Cortex, | neurons from | duration |
| | 2. Parietal | | Medial septum, | focus site in | reduction |
| | foci | | Cerebellum, or | projected brain | c. SUDEP |
| | 3. Occipital | | other | areas | frequency |
| | foci | | | | reduction |
| | | | | | d. QOL |
| | | | | | improvement |
| | | | | | e. Reduce |
| | | | | | invasiveness of |
| | | | | | neurostimulator |
| Movement | Parkinson's | Basal Ganglia: | Motor pathways | 1. Specific | a. Motor |
| disorders | disease | 1. STN | projections from | inhibition of STN | symptoms |
| | | 2. GPi + SNr | origin nucleus: | motor pathways | improvement |
| | | 3. Cortex | 1. GPi + SNr | to improve motor | (bradykinesia, |
| | | | 2. Thalamus | symptoms, | tremor, dystonia, |
| | | | 3. STN | without inhibition | etc.) |
| | | | | of other pathways | b. Reduction in |
| | | | | (e.g. limbic) | electric |
| | | | | 2. Specific | stimulation- |
| | | | | inhibition of | related adverse |
| | | | | GPi + SNr motor | effects |
| | | | | pathways to | |
| | | | | improve motor | |
| | | | | symptoms | |
| | | | | without inhibition | |
| | | | | of other pathways | |
| | | | | 3. Specific | |
| | | | | inhibition of the | |
| | | | | hyperdirect motor | |
| | | | | pathway to | |
| | | | | improve motor | |
| | | | | symptoms | |
| | | | | without inhibition | |
| | | | | of other pathways | |
| | Essential | 1. Thalamus - | 1. Motor | 1. Inhibition of | a. Tremor |
| | tremor | VIM | pathways | eOPN3 opsins | symptoms |
| | | 2. Cerebellum - | projections | in thalamic | improvement |
| | | Purkinje | from origin | neurons | b. Reduction in |
| | | cells (PC) | nucleus in the | projections in | electric |
| | | | cortex | the cortex, to | stimulation |
| | | | 2. Motor | eliminate | related adverse |
| | | | pathways from | tremor. | effects |
| | | | PCs to Deep | 2. Inhibition of | c. Reduction of |
| | | | Cerebellar Nuclei | eOPN3 opsins | invasiveness of |
| | | | (DCN) | in Purkinje | neurostimulator |
| | | | | Cells | device |
| | | | | projections in | |
| | | | | the DCN, to | |
| | | | | eliminate | |
| | | | | tremor. | |
| Pain | Trigeminal | Trigeminal | Spinal nucleus of | Inhibition of pain | 1. Pain intensity |
| | neuralgia | ganglion | Trigeminal Nerve | fibers of the | reduction |
| | | | | trigeminal nerve, | 2. Episode |
| | | | | without inhibition | duration |
| | | | | of touch, | reduction |
| | | | | proprioception, or | 3. Time to pain |
| | | | | motor functions. | reduction |
| | Migraine | Trigeminal | Dural nerve | Specific | 1. Pain intensity |
| | | ganglion | terminals | inhibition of | reduction |
| | | | | eOPN3- | 2. Episode |
| | | | | expressing | duration |
| | | | | trigeminal | reduction |
| | | | | ganglion cells | 3. Episode |
| | | | | projections in | frequency |
| | | | | anterior dura. | reduction |
| | | | | | 4. Time to pain |
| | | | | | reduction |
| | Occipital | C2 Dorsal Root | Posterior horn of | Inhibition of | 1. Pain intensity |
| | Migraine | Ganglion | Spinal Cord | eOPN3- | reduction |
| | | | | expressing C2 | 2. Episode |
| | | | | DRG neurons | duration |
| | | | | terminals at the | reduction |
| | | | | posterior horn of | 3. Episode |
| | | | | the spinal cord. | frequency |
| | | | | | reduction |
| | Lower back | T10-S2 DRGs | Posterior horn of | Inhibition of | 1. Pain intensity |
| | pain & | (according to | Spinal Cord | eOPN3- | reduction |
| | sciatica | patient's | | expressing DRG | 2. Episode |
| | | symptoms) | | neurons terminals | duration |
| | | | | at the posterior | reduction |
| | | | | horn of the spinal | 3. Pain |
| | | | | cord | medication |
| | | | | | reduction |
| | | | | | 4. Time to pain |
| | | | | | reduction |
| | Post | Corresponding | Posterior horn of | Inhibition of | 1. Pain intensity |
| | Herpetic | dermatome | Spinal Cord | eOPN3- | reduction |
| | neuralgia & | Dorsal Root | | expressing DRG | 2. Episode |
| | Phantom | Ganglion/ganglia | | neurons terminals | duration |
| | pain | | | at the posterior | reduction |
| | | | | horn of the spinal | 3. Pain |
| | | | | cord | medication |
| | | | | | reduction |
| | | | | | 4. Time to pain |
| | | | | | reduction |
| | CRPS | Corresponding | Posterior horn of | Inhibition of | 1. Pain intensity |
| | | Dorsal Root | Spinal Cord at | eOPN3- | reduction |
| | | Ganglions per | corresponding | expressing DRG | 2. Episode |
| | | limb affected | sites | neurons terminals | duration |
| | | | | at the posterior | reduction |
| | | | | horn of the spinal | 3. Pain |
| | | | | cord | medication |
| | | | | | reduction |
| | | | | | 4. Time to pain |
| | | | | | reduction |
| | Central pain | 1. Cingulate | 1. PAG | 1. Inhibition of | 1. Pain intensity |
| | | cortex | 2. Cingulate | eOPN3- | reduction |
| | | 2. Thalamus | cortex | expressing | 2. Episode |
| | | | | Cingulate | duration |
| | | | | cortex neurons | reduction |
| | | | | terminals at the | 3. Pain |
| | | | | PeriAqueductal | medication |
| | | | | Grey | reduction |
| | | | | 2. Inhibition of | 4. Time to pain |
| | | | | eOPN3- | reduction |
| | | | | expressing | |
| | | | | thalamic | |
| | | | | neurons | |
| | | | | terminals at the | |
| | | | | Cingulate | |
| | | | | cortex | |
| Sleep/ | Narcolepsy | Lateral | Midbrain - locus | Specific | 1. Sleep stability |
| wake cycle | & Insomnia | hypothalamus | coeruleus (LC) | inhibition of | and quality |
| | | | and raphe nuclei | Orexin neurons | 2. Cataplexy |
| | | | (Lateral | terminals in | episodes |
| | | | hypothalamus | midbrain (e.g. | frequency and |
| | | | Orexin neurons | LC, Raphe n.) to | duration |
| | | | terminals in the | induce stable | reduction |
| | | | midbrain) | sleep state at | 3. Wakefulness |
| | | | | night | maintenance |
| | | | | | stability increase |
| | | | | | (secondary) |
| Psychiatry | Obsessive | Nucleus | Prefrontal cortex | Inhibition of | 1. Decrease in |
| | Compulsive | Accumbens | | eOPN3 | Y-BOCS |
| | Disorder | | | expressing | severity scale |
| | | | | terminals of | 2. Reduction in |
| | | | | Nucleus | adverse effects |
| | | | | Accumbens | related to |
| | | | | neurons in the | electrical |
| | | | | PreFrontal cortex | stimulation |
| | | | | to alleviate | (Forgetfulness, |
| | | | | compulsive | word finding |
| | | | | behavior and | problems) |
| | | | | obsessive thought | 3. Decrease in |
| | | | | processes | medication |
| | | | | | intake |
| | Addiction | 1. Nucleus | 1. PreFrontal | Inhibition of | 1. Decrease in |
| | | Accumbens | Cortex | eOPN3 | addictive activity |
| | | 2. Striatum | 2. Nucleus | expressing | (drugs, alcohol, |
| | | | Accumbens | terminals of | smoking, other) |
| | | | | Nucleus | 2. Decrease in |
| | | | | accumbens | feelings of |
| | | | | neurons in the | craving |
| | | | | preFrontal cortex/ | |
| | | | | striatal neurons | |
| | | | | in the Nucleus | |
| | | | | accumbens to | |
| | | | | alleviate | |
| | | | | addictive | |
| | | | | symptoms and | |
| | | | | craving. | |
| Neuro- | Spasticity | 1. Anterior horn | 1. Muscles | 1. Inhibition of | 1. Muscle tone |
| muscular | | of spinal cord | (Nerves terminals | hyper-excitable | reduction |
| | | 2. Muscles | in muscles) | motor neurons in | 2. Pain reduction |
| | | (according to | 2. Posterior horn | the level of the | 3. Spasticity |
| | | symptoms) | of spinal cord | muscles to lower | medication |
| | | | (reflex afferents - | muscle tone | reduction |
| | | | e.g. spindle) | directly | 4. Contracture |
| | | | | 2. Inhibition of | severity |
| | | | | muscle reflex | reduction |
| | | | | afferents in the | |
| | | | | spinal cord to | |
| | | | | diminish hyper- | |
| | | | | excitable reflex | |
| | | | | circuit | |
| | Achalasia | Myenteric | Lower | Inhibition of | 1. Reduction of |
| | | (Auerbach) | Esophageal | eOPN3 | LES pressure in |
| | | plexus | sphincter | expressing | Manometry |
| | | (Cholinergic | externally or | terminals of | 2. Improvement |
| | | neurons) near | through the | Myenteric | in Barium |
| | | the Lower | esophageal lumen | postganglionic | swallowing test |
| | | Esophageal | | nerves in the | 3. improvement |
| | | Sphincter | | hyper excited | of Achalasia |
| | | (LES) or | | smooth muscle | symptoms |
| | | injection to the | | fibers of the LES | (reduction of |
| | | LES using retro | | during eating to | undigested food |
| | | AAV (to | | allow LES | regurgitaion, |
| | | transfect same | | relaxation and | heartburn, etc.) |
| | | neurons) | | food passage into | 4. Reduction in |
| | | | | the stomach, | reflux symptoms |
| | | | | while preventing | (as compared to |
| | | | | acid reflux when | post Heller's |
| | | | | not eating. | myotomy |
| | | | | | historical data). |
| | Urinary | Superior | Internal Urethral | Inhibition of | 1. Decrease of |
| | retention | hypogastric | Sphincter | eOPN3 | Internal Urethral |
| | | plexus | | expressing | Sphincter tone |
| | | (Noradrenergic | | terminals of NA | 2. Reduction of |
| | | (NA) neurons) | | Hypogastric | urine postvoid |
| | | or injection into | | nerve neurons in | residual volume |
| | | Internal | | the Internal | in bladder as |
| | | Urethral | | Urethral | seen in US scan |
| | | Sphincter using | | Sphincter to | 3. Reduction of |
| | | Retro AAV | | allow voiding of | abdominal pain |
| | | (targeting same | | bladder upon | |
| | | neurons) | | illuminating. | |
| Auto- | Ulcerative | 1. Insula | 1. Brain stem - | Inhibition of | 1. Plasma TNFα |
| Immune | Colitis | 2. Vagus nerve | Vagus nerve | eOPN3 | levels reduction |
| | | nuclei [e.g. the | nuclei (e.g. | expressing | 2. Plasma |
| | | dorsal motor | DMV, RVLM, | terminals of | inflammatory |
| | | nucleus of the | etc.) | insular cortical | markers levels |
| | | vagus (DMV), | 2. Colon | neurons in the | reduction |
| | | the vagal | | brain stem/ | 3. Symptoms |
| | | control center, | | terminals of | improvement |
| | | and the rostral | | DMV or RVLM | (abdominal pain, |
| | | ventrolateral | | in the colon in | Diarrhea, etc.). |
| | | medulla | | order to lower | |
| | | (RVLM)] | | TNFα-mediated | |
| | | | | inflammatory | |
| | | | | response in | |
| | | | | Ulcerative Colitis | |
| Neuro- | Appetite | Hypothalamus | Hypothalamus | Inhibition of | 1. Weight |
| endocrine | disorder | (ARC nucleus) | nuclei - PVN | AgRP neurons | reduction (or |
| | (e.g. Prader | | (Paraventricular | synapses with | slowing of |
| | Willi | | nucleus), PBN | POMC neurons | weight gain) |
| | Syndrome) | | (ParaBrachial | in the | 2. Appetite and |
| | & Obesity | | Nucleus) | hypothalamus or | food intake |
| | | | Or | BNST in order to | reduction |
| | | | BNST (BEd | reduce appetite | 3. BMI reduction |
| | | | Nucleus of Stria | mechanism and | |
| | | | Terminalis) | excessive eating | |
| | | | | (Hyperphagia) | |
| | | | | and obesity | |

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion. Generally, the nomenclature used herein and the laboratory procedures utilized in the present invention include molecular, biochemical, microbiological and recombinant DNA techniques.

### Example 1

### Treatment of Epilepsy

A mosquito (Anopheles stephensi)-derived homolog of the human encephalopsin protein (OPN3), a bistable type II opsin (i.e. remain bound to the retinal chromophore after illumination and display prolonged signal transduction following a single illumination pulse) that can be expressed on membranes of rat hippocampal neurons and most importantly in distal axonal presynaptic terminals by the addition of an ER export signal and membrane trafficking signal of a Kir2.1 protein was previously described International Patent Application Publication No. WO2020/188572, and referred to herein as “eOPN3”.

In an effort to control focal epileptic seizures through specific inhibition of projections from the epileptic focus, eOPN3 is administered to the seizure onset zone (also known as Epileptogenic focus) in epileptic animal models followed by illumination of the relevant terminals.

To this end, recombinant AAV vectors encoding eOPN3 are produced as described in International Patent Application Publication No. WO2020/188572. Briefly, a construct encoding the OPN3 opsin is subcloned into pAAV vectors under the CamKIIα promoter (targeting glutamatergic neurons) and in-frame with mScarlet at the C-terminus. A nucleic acid encoding the Kir2.1 membrane trafficking signal (KSRITSEGEYIPLDQIDINV, SEQ ID NO: 1) is added between the opsin and the mScarlet coding sequences and a nucleic acid encoding the Kir2.1 ER export signal (FCYENEV, SEQ ID NO: 2) is added following the C-terminus of mScarlet. The sequences of the eOPN3-mScarlet open reading frames are provided in SEQ ID NOs: 5-6 (the mScarlet nucleic acid sequence is provided in SEQ ID NO: 7).

The intrahippocampal Kainate rodent model, a focal-onset epilepsy model (see Lévesque M, et al. (20123) Neurosci Biobehav Rev. 37(10 Pt 2):2887-99; and Twele F et al. (2017) Epilepsia Open. 2(2):180-187), is used in this Example. Specifically, wild-type rodents are injected with Kainic acid unilaterally into the hippocampus. Seizures begin to appear gradually over a period of 2-3 months. Approximately 1 month following the Kainate injections, the rodents are stereotactically injected unilaterally into the same hippocampus with the eOPN3 encoding viral vector. Control animals are similarly injected with a control agent (AAV containing GFP fluorophore transgene) into the same target. Mice are allowed to recover for 6-8 weeks to allow for viral expression and intracellular transmission of the opsin to the synaptic terminals.

Following the aforementioned waiting period, the animals are implanted with a skull-mounted device that allows electrophysiological recording as well as illumination of terminals in the thalamus and/or Medial septum. The efficacy of illumination (amount, extent and/or duration) is then measured using either an open (continuous illumination for a set period of time) or a closed-loop (autonomous illumination operated by a seizure detection algorithm software) illumination protocols. The results are compared to no illumination periods and control animals. Endpoints measures include seizure frequency reduction, seizure duration reduction, seizure symptoms reduction, expression patterns, etc.

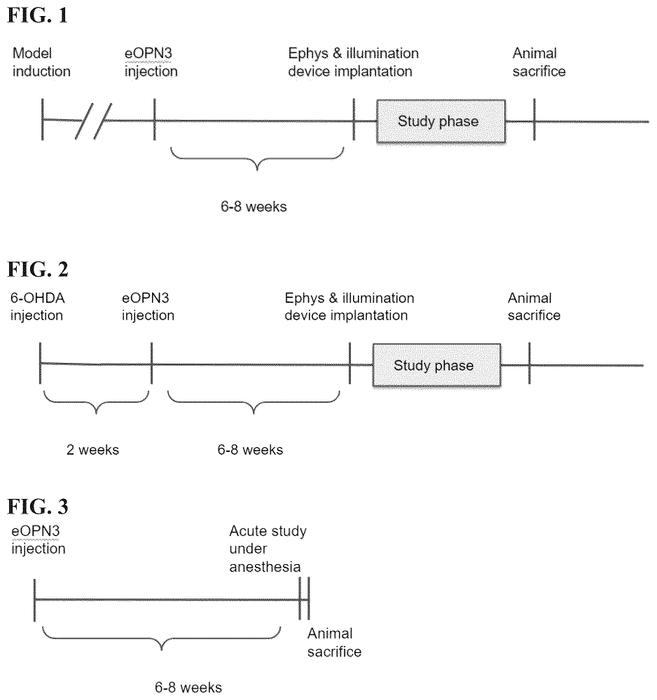

The experimental timeline and a summing outline table are provided in FIG. 1 and Table 2 hereinbelow.

TABLE 2
Outline table

| | | Site of | | |
|---|---|---|---|---|
| Indication | Agent | administration | Site of expression | Site of illumination |
| Focal onset epilepsy | AVV | Focus site | Focus site neurons - | Regions having projections |
| (e.g. Temporal Lobe | encoding | | cell bodies and | with the respective |
| epilepsy, frontal lobe | eOPN3 | | axon terminals | administration site |
| epilepsy, etc.) | | | | e.g. - Thalamus, Medial |
| | | | | septum |

### Example 2

### Treatment of Parkinson'S Disease

It is known that in Parkinson's disease, STN hyper-excitability plays a major role in motor symptoms including bradykinesia, tremor, dystonia and others. STN Deep Brain Stimulation (DBS) as well as lesioning techniques [Bergman H, et al. (1990) Science 249(4975): 1436-8] have been shown to improve motor symptoms; however, electrical stimulation is known to cause several serious adverse effects due to the unspecific nature of the treatment. These adverse effects include ‘area effects’ caused by the stimulation of nearby structures (e.g. Internal capsule fibers, Oculomotor nerve fibers, etc.) which may lead to a narrow therapeutic window, and ‘pathway effects’ caused by the non-specific inhibition of STN projections to unrelated brain areas (e.g. limbic, cognitive and associative pathways).

In an effort to achieve effective and precise STN inhibition, alleviate motor symptoms and avoid ‘area’ and/or ‘pathway’ effects, recombinant AAV vectors encoding eOPN3 (produced as described in Example 1 hereinabove) are administered to the STN, GPi and/or SNr of Parkinson animal models followed by illumination of the relevant terminals.

To this end, wild type rodents are injected with 6-OHDA unilaterally into the basal ganglia (usually into the striatum or MFB areas) to induce unilateral Parkinsonism, which is characterized by contralateral motor symptoms (e.g. bradykinesia, weakness, etc.). Subsequently, the rodents are stereotactically injected with the eOPN3 encoding viral vector unilaterally into 1) the ipsilateral STN or 2) the GPi (in rodents also known as EPN) and SNr. Control animals are similarly injected with a control agent (AAV containing GFP fluorophore transgene) into the same target. Mice are allowed to recover for 6-8 weeks to allow for viral expression and intracellular transmission of the opsin to the synaptic terminals [in the GPi & SNr, as well as other connections of the STN in 1) and the thalamus in 2).

Following the aforementioned waiting period, the animals are implanted with a skull-mounted device that allows electrophysiological recording as well as illumination. The illumination protocols comprise periods of no illumination followed by periods of continuous illumination, and a second period of no illumination. The efficiency of illumination on alleviating Parkinsonian motor symptoms is measured while monitoring the animal movement in open field and on Rotarod. Endpoints measures include distance traveled, velocity, CW and CCW rotations, the electrophysiological activity of the STN, expression patterns, etc.

The experimental timeline and a summing outline table are provided in FIG. 2 and Table 3 hereinbelow.

TABLE 3
Outline table

| | | Site of | | |
|---|---|---|---|---|
| Indication | Agent | administration | Site of expression | Site of illumination |
| Parkinson's disease | AVV | STN | STN, GPi, SNr, | GPi + SNr |
| | encoding | | GPe, Cortex etc. | |
| | eOPN3 | GPi + SNr | Thalamus, GPe, | Thalamus |
| | | | STN | |

### Example 3

### Treatment of Migraines

The Trigeminal nerve is responsible for the sensory innervation of the anterior ⅔ of the head. Thus, many headache syndromes are correlated with pathologies in the Trigeminal nerve including for example Trigeminal Neuralgia, Cluster headache, and Migraines. Trigeminal Neuralgia is characterized by paroxysmal attacks of very severe pain, which are usually non-responsive to pain medication. Migraines are headaches that are generated by a cascade of inflammatory events in the dura, thought to be initiated by trigeminal nerve dendrite terminals that release neuropeptides such as CGRP, which in turn, initiates inflammatory response which leads to dilation of blood vessels and pain.

It is important to note that pain is just one type of sensation carried by the Trigeminal nerve, which is also responsible to convey other sensory inputs like touch, temperature, vibration, proprioception, etc. Therefore, treatments that aim to ablate or anesthetize the Trigeminal nerve nonspecifically may cause secondary damage due to loss of important sensory input. An example of that effect is the loss of the Corneal reflex (that helps close the eyelid when the eye is dry, or being touched), which may lead to drying of the cornea and long-term damage to the eye.

In an effort to achieve effective and precise inhibition of pain fibers of the trigeminal nerve, recombinant AAV vectors encoding eOPN3 (produced as described in Example 1 hereinabove) are administered to the trigeminal ganglion of an animal model of acute headaches and migraines followed by illumination of the relevant terminals in the dura. More specifically, this animal pain model is used to assess whether illumination of synaptic terminals of nociceptive axons of the Trigeminal nerve expressing eOPN3 in the dura, inhibits their responses to mechanical and chemical stimulation of the dura, and to cortical spreading depression (the neural events that mediate migraine aura in patients).

To this end, wild type rodents are injected with the eOPN3 encoding viral vector stereotactically into the Trigeminal Ganglion. Control animals are similarly injected with a control agent (AAV containing GFP fluorophore transgene) into the same target. Mice are allowed to recover for 6-8 weeks to allow for viral expression and intracellular transmission of the opsin to the synaptic terminals.

Following the aforementioned waiting period, the animals are anesthetized and prepared for the study with craniotomy. Expression of eOPN3 in the dura and spine is validated with a microscope (specifically, following craniotomy the dura is exposed and the fluorescence tag can be detected using a confocal microscope), and electrodes are placed. First, baseline responses are evaluated with brief mechanical [dural indentation with Von Frey Hair monofilaments (VFH)] and chemical (Kcl) stimulations of the dura, in the dark, while recording electrophysiologic activity. Then, the same responses are evaluated during illumination of the dura area being studied. In parallel to both, an assessment of sensory input from the skin in an unilluminated area of the head is evaluated to show the effect is focused on the illuminated area. In addition, Cortical Spreading depression (CSD) is evaluated by comparing 1-hour recording of neuronal activity in baseline, and then 1 hour of activity following induced CSD. After performing these recordings in the dark, the same is repeated with illumination. In both cases, sensory activity from unilluminated areas is assessed in parallel. Subsequently, the animals are sacrificed and histological confirmation of expression in the dura and the Trigeminal ganglion is evaluated.

The experimental timeline and a summing outline table are provided in FIG. 3 and Table 4 hereinbelow.

TABLE 4
Outline table

| | | Site of | | |
|---|---|---|---|---|
| Indication | Agent | administration | Site of expression | Site of illumination |
| Migraines | AVV | Trigeminal | The Trigeminal | Dural nerve terminals |
| Trigeminal Neuralgia | encoding | ganglion | ganglion + Axon | Spinal nucleus of Trigeminal |
| | eOPN3 | | and dendrites | Nerve |
| | | | terminals | |

### Example 4

### Treatment of Peripheral Pain

Uncontrolled pain is one of the biggest unmet needs in medicine. More than 100M Americans suffer from pain, and the total incremental costs of healthcare due to pain ranged from $261 to $300 billion USD [Gaskin et al. J. Pain. 2012]. Pain is notoriously undertreated, leading to an infamous “plague” of opiate use and subsequent addiction. Several pain syndromes are characterized by aberrant pain signaling that does not represent a correct sensation of pain (e.g. —neuropathies, phantom pain, PNH, sciatica, abdominal & bladder pain, and others) and are unresponsive to many available pain medications; however, many are reactive to local anesthesia (e.g. nerve blocks done with Lidocaine or Botox).

The present inventors suggest that expressing eOPN3 in targeted Dorsal Root Ganglion (DRG) nerves corresponding with involved dermatomes will allow to achieve a “switchable nerve block”, which could be operated by the patient in an online manner. Using AAVs with neurotropism to unmyelinated fibers may allow a specific effect on pain without inhibition of other sensory neurons. This could be achieved by injection of the AAV into the peripheral nerve or targeted DRGs and illumination of projections in the posterior horn of the spinal cord.

To this end, wild type rodents are injected intrathecally in the lumbar region with the eOPN3 encoding viral (produced as described in Example 1 hereinabove). This administration method leads to a wide expression in the DRGs, throughout the spine. Control animals are similarly injected with a control agent (AAV containing GFP fluorophore transgene) in the same method. After a waiting period of 6 weeks, the rodents are evaluated behaviorally and electro physiologically in response to painful stimuli (hot plate) in the dark and under external illumination to the posterior horn of the spinal cord (fur will be shaved in the illumination area in the lower back-hind leg DRGs: L2-L5). The same testing is repeated 14 weeks after the injection. At the end of each period of the study (i.e. 6 weeks and 14 weeks following injection), animals are sacrificed and tissues are evaluated histologically.

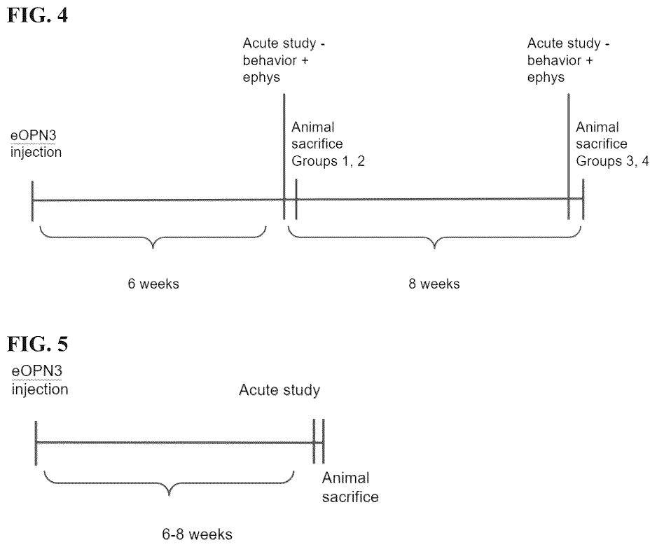

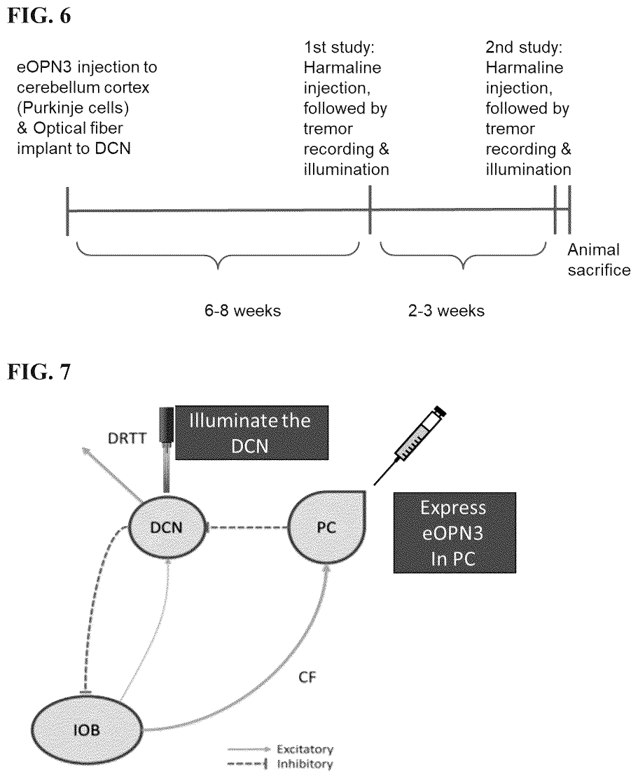

The experimental timeline and a summing outline table are provided in FIG. 4 and Table 5 hereinbelow.

TABLE 5
Outline table

| | | Site of | Site of | Site of |
|---|---|---|---|---|
| Indication | Agent | administration | expression | illumination |
| Peripheral pain: Neuropathic, | AVV | Peripheral nerves | DRG cell bodies | Posterior horn of |
| inflammatory or nociceptive | encoding | or Dorsal Root | and axon | Spinal Cord |
| pain (including: Trigeminal | eOPN3 | Ganglion (DRG) | terminals | |
| neuralgia, Occipital | | according to | | |
| Migraines, Cluster headache, | | patient's | | |
| Tension headache, Phantom | | symptoms, (e.g. | | |
| pain, CRPS, Sciatica, | | C2 DRG, T10-S2 | | |
| Postherpetic neuralgia, cancer | | DRGs, | | |
| pain, chemotherapy-induced | | Corresponding | | |
| neuropathy, Diabetic | | dermatome DRG) | | |
| neuropathy, postoperative | | | | |
| pain, Abdominal pain, | | | | |
| Bladder pain, and more) | | | | |

### Example 5

### Treatment of Sleep Disorders

The hypothalamus monitors body homeostasis and regulates various behaviors such as feeding, thermogenesis, and sleeping. Orexins (also known as hypocretins) were identified as endogenous ligands for two orphan G-protein-coupled receptors in the lateral hypothalamic area. Orexins activate orexin neurons, monoaminergic and cholinergic neurons in the hypothalamus/brainstem regions, to maintain a long, consolidated awake period [Inutsuka A, Yamanaka A. Front Endocrinol, 2013]. Loss of Orexin's function leads to narcolepsy, which is a condition characterized by an unstable wakefulness state, causing patients to fall asleep abruptly and suddenly during awake hours. Treatments for this condition focus on improving sleep during night time or improving wakefulness during the day.

The present inventors suggest that expressing eOPN3 under a specific Orexin promoter, will allow a switchable inhibitory effect on Orexin neurons leading to a stable sleep during sleeping periods, which can be applicable for narcolepsy and other sleep disorders.

To this end, wild type rodents are injected stereotactically into the lateral hypothalamus, bilaterally, with an eOPN3 encoding viral under a specific Orexin promoter (produced as described in Example 1 hereinabove but under the Orexin promoter (described in e.g. Moriguchi T et al. (2002) J Biol Chem. 277(19): 16985-92, instead of the CamKIIα promoter). Control animals are similarly injected with a control agent (AAV containing GFP fluorophore transgene) into the same target. After a waiting period of 6-8 weeks, the quality of sleep in the rodents in response to external transcranial illumination as compared to no illumination is evaluated. Specifically, behavioral and electrophysiological measurements include sleep-wake quantities (episode frequency, duration, average), sleep quality (power spectrum analysis), local LFPs activities (power spectrum analysis, slow wave & spindle detection), and assessment of circuit coherences. Following, the animals are sacrificed and histological confirmation of eOPN3 expression in Orexin neurons is performed.

The experimental timeline and a summing outline table are provided in FIG. 5 and Table 6 hereinbelow.

TABLE 6
Outline table

| | | Site of | | |
|---|---|---|---|---|
| Indication | Agent | administration | Site of expression | Site of illumination |
| Narcolepsy | AVV | Lateral | Hypothalamus - cell | Midbrain - locus |
| Insomnia | encoding | hypothalamus | bodies and | coeruleus (LC) and raphe |
| REM Behavior | eOPN3 | | terminals, Midbrain | nuclei |
| Disorder | | | (axon terminals), | (Lateral hypothalamus |
| | | | Cortex (axon | Orexin neurons terminals |
| | | | terminals) | in the midbrain) |

### Example 6

### Treatment of Chronic Essential Tremor

Essential Tremor (ET) is the most common movement disorder worldwide, with an estimated prevalence worldwide of 1% overall and approximately 5% in adults over the age of 60 years (Louis E D, Ferreira J J. Mov Disord. 2010; 25(5):534). It classically involves the hands and is brought out by arm movement and sustained antigravity postures, affecting common daily activities such as writing, drinking from a glass, and handling eating utensils. Pathological activities within Cerebellar-Thalamic-Cortical pathways are thought to be responsible for this pathology (Steven Bellows, Joohi Jimenez-Shahed, International Review of Neurobiology, Academic Press, Volume 163, 2022.). Current treatment for ET includes surgical interventions like DBS (targeted at the VIM nucleus of the thalamus or the STN) or lesioning interventions like Focused-Ultrasound (FUS) ablation of VIM nucleus. While considered effective for refractory ET, these treatments are associated with frequent and many times severe adverse effects, such as speech impairments, dysarthria, and other side effects, associated by the imprecise electrical stimulation affecting surrounding structures or the irreversible destruction of tissue caused by FUS heating.

### Materials and Methods

In an effort to achieve effective and precise cerebellar-thalamic inhibition, alleviate tremor and avoid ‘area’ effects, in a reversible manner, recombinant AAV vectors encoding eOPN3 (produced as described in Example 1 hereinabove) were administered to the cerebellar cortex, targeting Purkinje Cells (PC), followed by illumination of PC terminals in the Deep Cerebellar Nucleus (DCN).

To this end, wild type mice were injected with eOPN3 encoding viral vectors bilaterally into the cerebellar cortex, and optical fibers were implanted bilaterally above the DCN. After a waiting period of 6-8 weeks for expression and opsin transport to presynaptic terminals, the mice were injected systemically with Harmaline, to induce tremor. The Harmaline-induced tremor lasts for approx. 3 hours and exacerbated by movement. Following Harmaline injection, the mice were put into a pressure plate for tremor measurement. In addition, an accelerometer was adhered to the mouse's back, and the optical fibers were connected to a light source (LED, Green, 525 nm, ˜10 mW).

The illumination protocol comprised periods of no illumination to allow recording of baseline tremor, followed by illumination for 4-5 minutes, in which the tremor was relieved, followed by a second period of no illumination to record the recovery of the tremor.

The efficiency of illumination on alleviating tremor was measured while monitoring the animal movement in open field. Endpoints measures include tremor intensity, frequency, kinetics of on and off times, etc.

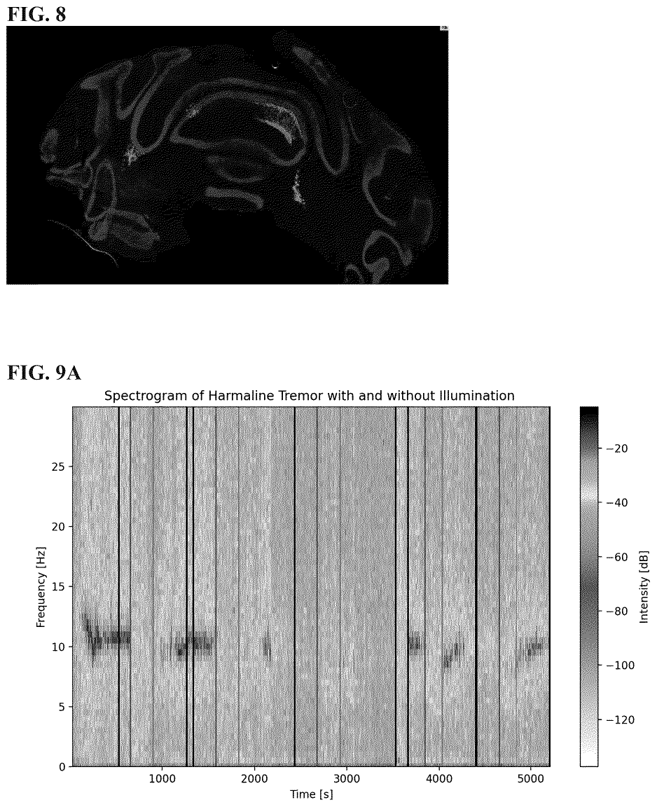

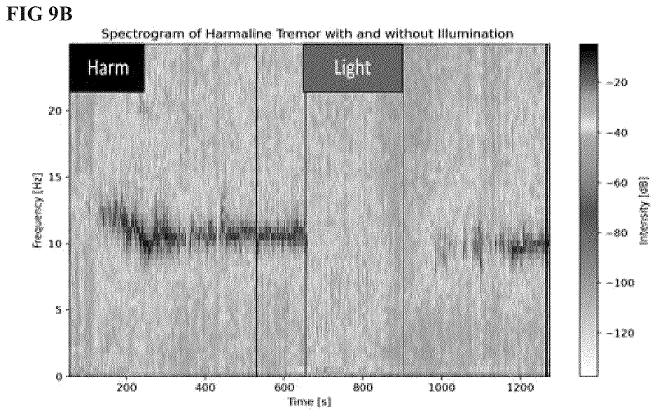

Results: Harmaline injection induces tremor symptoms that lasts ˜3 hours, and is exacerbated by movement. Baseline recordings showed intense tremor symptoms that were picked up by the accelerometer on the mouse back (FIGS. 9A-B). Illumination of presynaptic terminals of Purkinje cells arriving from the cerebellar cortex to the DCN, effectively diminished tremor intensity (FIGS. 9A-B). Illumination cessation resulted in the return of the tremor after 1-2 minutes (FIGS. 9A-B). These results are consistent with eOPN3 known kinetics and show the efficacy of this specific pathway modulation in treating tremor.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a disease associated with pathologic neuronal cells characterized by aberrant excitability in a subject in need thereof, the method comprising:
(a) administering into a first neural region of the subject a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; and
(b) exposing a second neural region of said subject to light in a wavelength that activates said bistable type II opsin, wherein said second neural region comprises axon or dendrite terminals of said pathologic neuronal cells associated with a symptom of said disease,
wherein said first neural region and said second neural region are distinct, and
wherein when said disease is epilepsy said first neural region is not a thalamus nucleus,
thereby treating the disease in the subject.

2. A method of treating a disease associated with pathologic neuronal cells characterized by aberrant excitability in a subject in need thereof having been administered into a first neural region a therapeutically effective amount of a polynucleotide encoding a bistable type II opsin attached to a heterologous ER export signal and/or membrane trafficking signal which enables trafficking of said bistable type II opsin to axon and/or dendrite terminals, wherein said first neural region comprises cell bodies of said pathologic neuronal cells; the method comprising exposing a second neural region of said subject to light in a wavelength that activates said bistable type II opsin, wherein said second neural region comprises axon or dendrite terminals of said pathologic neuronal cells associated with a symptom of said disease, wherein said first neural region and said second neural region are distinct, and wherein when said disease is epilepsy said first neural region is not a thalamus nucleus, thereby treating the disease in the subject.

3. The method of claim 1, wherein said aberrant excitability comprises hyper-excitability.

4. The method of claim 1, wherein said aberrant excitability comprises mis-synchronization.

5. The method of claim 1, wherein said aberrant excitability comprises spatial or temporal aberrancy.

6. The method of claim 1, wherein said disease is selected from the group consisting of epilepsy, Parkinson's disease, essential tremor, pain, spasticity, Achalasia, obsessive-compulsive disorder (OCD), addiction, appetite disorder, autoimmune disorder, narcolepsy and insomnia.

7. The method of claim 1, wherein said disease, said first neural region and said second neural region are as listed in Table 1.

8. The method of claim 1, wherein said administering is by a stereotactic injection.

9. The method of claim 1, wherein said polynucleotide is packed in a viral vector.

10. The method of claim 1, wherein said bistable type II opsin is selected from the group consisting of OPN3, OPN4, OPN5, LcPP, DrPP2, TrPP2, parapinopsin, PdCO, TMT and peropsin.

11. The method of claim 1, wherein said bistable type II opsin is OPN3.

12. The method of claim 11, wherein said OPN3 is mosquito OPN3 (MosOpn3).

13. The method of claim 1, wherein said ER export signal and/or said membrane trafficking signal is of a protein expressed in neuronal cells; and optionally wherein said protein is a Kir2.1.

14. The method of claim 1, wherein an amino acid sequence of said ER export signal comprises SEQ ID NO: 2; and/or wherein an amino acid sequence of said membrane trafficking signal comprises SEQ ID NO: 1.

15. The method of claim 1, wherein said exposing is effected at least 6 or 8 weeks following said administering.

16. The method of claim 1, wherein said exposing comprises repeated illumination independent of detection of an acute symptom of said disease.

17. The method of claim 1, wherein said exposing is effected upon detection of an acute symptom of said disease.

18. The method of claim 1, wherein said exposing is effected using a skull-mounted device, an intracranial device or a device implanted along the spine.

19. The method of claim 1, wherein said exposing is effected using a device external to the subject.

20. The method of claim 1, wherein said wavelength is 450-650 nm or 350-670 nm.


SEQUENCE LISTING (44 sequences)

SEQ ID NO: 1 - protein, 20 aa, synthetic construct
  REGION 1..20: note = Membrane trafficking signal
KSRITSEGEYIPLDQIDINV

SEQ ID NO: 2 - protein, 7 aa, synthetic construct
  REGION 1..7: note = ER EXPORT SIGNAL
FCYENEV

SEQ ID NO: 3 - skipped

SEQ ID NO: 4 - protein, 6 aa, synthetic construct
  REGION 1..6: note = Linker sequence
PRARDP

SEQ ID NO: 5 - protein, 607 aa, synthetic construct
  REGION 1..607: note = eOPN3-mScarlet open reading frame AA sequence
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMVTETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 6 - DNA, 1824 bp, synthetic construct
  misc_feature 1..1824: note = eOPN3 nucleic acid sequence
  source 1..1824: mol_type = other DNA
atgtacgacgcccccaacgacgtggccagcagcgtggccgactacgaggacctgatggccccctgggcctacaacgccgccgccatcaccctgttcttcatcggcttcttcggcttcttcctgaacctgttcgtgatcgccctgatgagcaaggacatgcagctgtggacccccatgaacatcatcctgttcaacctggtgtgcagcgacttcagcgtgagcatcatcggcaaccccctgaccctgaccagcgccatcagccacaggtggatcttcggcaggaccctgtgcgtggcctacggcttcttcatgagcctgctgggcatcaccagcatcaccaccctgaccgtgctgagctacgagaggtactgcctgatcagcaggcccttcagcagcaggaacctgagcaggaagggcgccttcctggccatcttcttcatctggggctacagcttcgccctgaccagcccccccctgttcggctggggcgcctacgtgcaggaggccgccaacatcagctgcagcgtgaactgggagagccagaccaagaacgccaccacctacatcatcttcctgttcgtgttcggcctggtggtgcccctgatcgtgatcgtgtacagctacaccaacatcatcgtgtacatgaggaggaacagcgccagggtgggcaggatcaacagggccgagcagagggtgaccagcatggtggccgtgatgatcgtggccttcatggtggcctggaccccctacgccatcttcgccctgatcgagcagttcggcccccccgagctgatcggccctggactggctgtgctgcctgctctgatcgccaagagcagcatctgctacaaccccatcatctacgtgggcatgaacacccagttcagggccgccttcaccagggtgaggaacaagggcggcgtgcccaccgccgaccagaacaccaccaccatgcagagggagctgaccaagagcagcagggacatggtgaccgagaccagccaggtggctcctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

SEQ ID NO: 7 - DNA, 696 bp, synthetic construct
  misc_feature 1..696: note = mScarlet NA sequence
  source 1..696: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 8 - protein, 429 aa, synthetic construct
  REGION 1..429: note = mosopn3 aa sequence
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMVECSFDFCRKKNRFKISLVKPTAPLAVVDVSSSSHPGKVTSRSPLDQTVLNEMNDEERGRERSGAGYAGSRFVRPDFELSVINSGKSILIKSKNFRSNLL

SEQ ID NO: 9 - protein, 330 aa, synthetic construct
  REGION 1..330: note = mosopn3 mutant without the last 99 aa
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMV

SEQ ID NO: 10 - protein, 288 aa, synthetic construct
  REGION 1..288: note = PufTMT amino acid sequence
MSRTGHTVVAVMLGTILLAGVFGNSVVFLVFVKYRSLRTPINLILLNISLSDILVCVFGTPLSFAASLKGRWLLGERGCEWYGFANSLFGIVSLVSLSVLSYERYTVVLQPTQVDVSYFRKAWFCVGGSWLYALFWTLPPLLGWSRYGPEGPGTMCSVQWHLRSPANISYVLCLFIFCLLLPLVVMVYSYGRIWVAVRRQHCAQSHLEAGRINLLTAQRREQHILWMVLSMVSCYMLCWMPYGIIALVATLGRLGPISPAVSVVPSILAKFSTVVNPVIYMFFNNQVR

SEQ ID NO: 11 - protein, 6 aa, synthetic construct
  REGION 1..6: note = ER EXPORT SIGNAL
  SITE 2: note = misc_feature - Xaa can be any naturally occurring amino acid
FXYENE

SEQ ID NO: 12 - skipped

SEQ ID NO: 13 - protein, 5 aa, synthetic construct
  REGION 1..5: note = ER EXPORT SIGNAL
VKESL

SEQ ID NO: 14 - protein, 5 aa, synthetic construct
  REGION 1..5: note = ER EXPORT SIGNAL
VLGSL

SEQ ID NO: 15 - protein, 16 aa, synthetic construct
  REGION 1..16: note = ER EXPORT SIGNAL
NANSFCYENEVALTSK

SEQ ID NO: 16 - protein, 26 aa, synthetic construct
  REGION 1..26: note = MEMBRANE TRAFFICKING SIGNAL
MDYGGALSAVGRELLFVTNPVVVNGS

SEQ ID NO: 17 - protein, 26 aa, synthetic construct
  REGION 1..26: note = MEMBRANE TRAFFICKING SIGNAL
AGHSNSMALFSFSLLWLCSGVLGTEF

SEQ ID NO: 18 - protein, 23 aa, synthetic construct
  REGION 1..23: note = MEMBRANE TRAFFICKING SIGNAL
MGLRALMLWLLAAAGLVRESLQG

SEQ ID NO: 19 - protein, 18 aa, synthetic construct
  REGION 1..18: note = MEMBRANE TRAFFICKING SIGNAL
MRGTPLLLVVSLFSLLQD

SEQ ID NO: 20 - DNA, 864 bp, synthetic construct
  misc_feature 1..864: note = Codon optimized nucleic acid sequence of the MosOpn3
  source 1..864: mol_type = other DNA
atgagcaggaccggccacaccgtggtggccgtgatgctgggcaccatcctgctggccggcgtgttcggcaacagcgtggtgttcctggtgttcgtgaagtacaggagcctgaggacccccatcaacctgatcctgctgaacatcagcctgagcgacatcctggtgtgcgtgttcggcacccccctgagcttcgccgccagcctgaagggcaggtggctgctgggcgagaggggctgcgagtggtacggcttcgccaacagcctgttcggcatcgtgagcctggtgagcctgagcgtgctgagctacgagaggtacaccgtggtgctgcagcccacccaggtggacgtgagctacttcaggaaggcctggttctgcgtgggcggcagctggctgtacgccctgttctggaccctgcctcctctgctgggctggagcaggtacggccccgagggccctggcaccatgtgcagcgtgcagtggcacctgaggagccccgccaacatcagctacgtgctgtgcctgttcatcttctgcctgctgctgcccctggtggtgatggtgtacagctacggcaggatctgggtggccgtgaggaggcagcactgcgcccagagccacctggaggccggcaggatcaacctgctgaccgcccagaggagggagcagcacatcctgtggatggtgctgagcatggtgagctgctacatgctgtgctggatgccctacggcatcatcgccctggtggccaccctgggcaggctgggccccatcagccccgccgtgagcgtggtgcccagcatcctggccaagttcagcaccgtggtgaaccccgtgatctacatgttcttcaacaaccaggtgagg

SEQ ID NO: 21 - DNA, 990 bp, synthetic construct
  misc_feature 1..990: note = Alternative Codon optimized nucleic acid sequence of the MosOpn3
  source 1..990: mol_type = other DNA
atgtacgacgcccccaacgacgtggccagcagcgtggccgactacgaggacctgatggccccctgggcctacaacgccgccgccatcaccctgttcttcatcggcttcttcggcttcttcctgaacctgttcgtgatcgccctgatgagcaaggacatgcagctgtggacccccatgaacatcatcctgttcaacctggtgtgcagcgacttcagcgtgagcatcatcggcaaccccctgaccctgaccagcgccatcagccaccgctggatcttcggccgcaccctgtgcgtggcctacggcttcttcatgagcctgctgggcatcaccagcatcaccaccctgaccgtgctgagctacgagcgctactgcctgatcagccgccccttcagcagccgcaacctgagccgcaagggcgccttcctggccatcttcttcatctggggctacagcttcgccctgaccagcccccccctgttcggctggggcgcctacgtgcaggaggccgccaacatcagctgcagcgtgaactgggagagccagaccaagaacgccaccacctacatcatcttcctgttcgtgttcggcctggtggtgcccctgatcgtgatcgtgtacagctacaccaacatcatcgtgtacatgcgccgcaacagcgcccgcgtgggccgcatcaaccgcgccgagcagcgcgtgaccagcatggtggccgtgatgatcgtggccttcatggtggcctggaccccctacgccatcttcgccctgatcgagcagttcggcccccccgagctgatcggccccggcctggccgtgctgcccgccctgatcgccaagagcagcatctgctacaaccccatcatctacgtgggcatgaacacccagttccgcgccgccttcacccgcgtgcgcaacaagggcggcgtgcccaccgccgaccagaacaccaccaccatgcagcgcgagctgaccaagagcagccgcgacatggtg

SEQ ID NO: 22 - DNA, 864 bp, synthetic construct; identical to SEQ ID NO: 20
  misc_feature 1..864: note = Codon optimized nucleic acid sequence of the PufTMT
  source 1..864: mol_type = other DNA

SEQ ID NO: 23 - DNA, 864 bp, synthetic construct
  misc_feature 1..864: note = Alternative Codon optimized nucleic acid sequence of the PufTMT
  source 1..864: mol_type = other DNA
atgagccgcaccggccacaccgtggtggccgtgatgctgggcaccatcctgctggccggcgtgttcggcaacagcgtggtgttcctggtgttcgtgaagtaccgcagcctgcgcacccccatcaacctgatcctgctgaacatcagcctgagcgacatcctggtgtgcgtgttcggcacccccctgagcttcgccgccagcctgaagggccgctggctgctgggcgagcgcggctgcgagtggtacggcttcgccaacagcctgttcggcatcgtgagcctggtgagcctgagcgtgctgagctacgagcgctacaccgtggtgctgcagcccacccaggtggacgtgagctacttccgcaaggcctggttctgcgtgggcggcagctggctgtacgccctgttctggaccctgccccccctgctgggctggagccgctacggccccgagggccccggcaccatgtgcagcgtgcagtggcacctgcgcagccccgccaacatcagctacgtgctgtgcctgttcatcttctgcctgctgctgcccctggtggtgatggtgtacagctacggccgcatctgggtggccgtgcgccgccagcactgcgcccagagccacctggaggccggccgcatcaacctgctgaccgcccagcgccgcgagcagcacatcctgtggatggtgctgagcatggtgagctgctacatgctgtgctggatgccctacggcatcatcgccctggtggccaccctgggccgcctgggccccatcagccccgccgtgagcgtggtgcccagcatcctggccaagttcagcaccgtggtgaaccccgtgatctacatgttcttcaacaaccaggtgcgc

SEQ ID NO: 24 - protein, 5 aa, synthetic construct
  REGION 1..5: note = repetetive amino acid sequence motif
EAAAK

SEQ ID NO: 25 - protein, 354 aa, synthetic construct
  REGION 1..354: note = PdCO2 aa sequence
MDGENLTIPNPVTELMDTPINSTYFQNLNAETDGGNHYIYNAFTATDYNICAAYLFFIACLGVSLNVLVLVLFIKDRKLRSPNNFLYVSLALGDLLVAVFGTAFKFIITARKTLLREEDGFCKWYGFITYLGGLAALMTLSVIAFVRCLAVLRLGSFTGLTTRMGVAAMAFIWIYSLAFTLAPLLGWNHYIPEGLATWCSIDWLSDETSDKSYVFAIFIFCFLVPVLIIVVSYGLIYDKVRKVAKTGGSVAKAEREVLRMTLLMVSLFMLAWSPYAVICMLASFGPKDLLHPVATVIPAMFAKSSTMYNPLIYVFMNKQFRRSLKVLLGMGVEDLNSESERATGGTATNQVAAT

SEQ ID NO: 26 - DNA, 1062 bp, synthetic construct
  misc_feature 1..1062: note = PdCO2 na sequence
  source 1..1062: mol_type = other DNA
atggacggggagaacttgacgattccaaatcctgtcaccgagttgatggataccccaatcaacagcacgtatttccaaaaccttaatgcggaaacggacggcggaaatcattacatctacaatgccttcacagccaccgattacaacatttgtgcagcttaccttttcttcattgcttgtctgggagtcagtctcaacgttttggtgttggtcctctttatcaaagacagaaagttgaggagccccaataacttcctgtatgtaagtctcgctctcggggatctgttggttgcagttttcggcaccgcatttaagttcattattacagctcgcaaaaccctgttgcgagaagaagacggtttctgcaaatggtacggcttcataacatacctcggcgggctcgcggcactgatgaccctgagcgttattgcttttgttcgttgtctggccgtgctgcgtctcggctccttcacaggtttgacaacgcggatgggcgtggcggcgatggctttcatttggatctactccctggcattcacactcgcgcctctccttgggtggaatcactacataccggaaggtttggccacttggtgtagtatcgactggctttccgatgaaacatccgataagtcctacgtgtttgctattttcatcttctgctttcttgtccctgttctcattatcgtcgtgtcatacgggctgatctacgataaggttcggaaggtagcgaaaacaggcggttctgttgccaaagcagaacgcgaagtactgcgcatgacgctgctgatggttagcctgttcatgcttgcctggagtccctacgctgttatctgcatgctcgctagcttcggccctaaagacctgctccatccagtggccacagtgattcctgccatgtttgcaaagtcttctacgatgtataaccctcttatctatgtgttcatgaacaaacaattcaggcggagccttaaagttcttctcggaatgggagtagaggacctgaactccgagagtgagagagcaactggtggtacagccacgaatcaagtcgccgcgacg

SEQ ID NO: 27-28 - skipped (2 entries)

SEQ ID NO: 29 - protein, 343 aa, synthetic construct
  REGION 1..343: note = LcPP aa sequence
MENLTSLDLLPNGEVPLMPRYGFTILAVIMAVFTIASLVLNSTVVIVTLRHRQLRHPLNFSLVNLAVADLGVTVFGASLVVETNAVGYFNLGRVGCVIEGFAVAFFGIAALCTIAVIAVDRFVVVCKPLGTLMFTRRHALLGIAWAWLWSFVWNTPPLFGWGSYELEGVRTSCAPDWYSRDPANVSYITSYFAFCFAIPFLVIVVAYGRLMWTLHQVAKLGMGESGSTAKAEAQVSRMVVVMVVAFLVCWLPYALFAMIVVTKPDVYIDPVIATLPMYLTKTSTVYNPIIYIFMNRQFRDCAVPFLLCGRNPWAEPSSESATAASTSATSVTLASAPGQVSPS

SEQ ID NO: 30 - DNA, 1029 bp, synthetic construct
  misc_feature 1..1029: note = LcPP na sequence
  source 1..1029: mol_type = other DNA
atggaaaatctgacttccctggatctgctgcccaacggagaggtcccactgatgccccggtacggctttactattctggctgtgattatggccgtgttcaccatcgcaagtctggtcctgaactcaactgtggtcattgtgaccctgcgacaccgacagctgaggcatcctctgaacttttccctggtgaatctggctgtcgcagacctgggcgtgacagtcttcggagcttctctggtggtcgagactaacgcagtggggtactttaatctgggacgcgtggggtgcgtcatcgaagggttcgccgtggctttctttggcattgccgctctgtgcaccatcgctgtgattgcagtcgatcgatttgtggtggtgtgcaagcccctgggaaccctgatgttcacaaggagacacgcactgctgggaatcgcatgggcatggctgtggagcttcgtgtggaacacaccccctctgttcggctggggaagctacgagctggaaggagtgagaactagctgcgctcctgactggtattcccgggaccccgccaacgtgagctacatcacatcttatttcgcattttgtttcgccatccccttcctggtcatcgtcgtggcttacggccggctgatgtggactctgcatcaggtggccaagctggggatgggcgagtctggaagtaccgctaaagcagaagcccaggtgagtcgcatggtcgtggtcatggtggtcgcctttctggtctgttggctgccctatgccctgttcgctatgatcgtggtcaccaagcctgacgtgtacatcgatccagtcattgccacactgcccatgtatctgaccaaaacaagcaccgtgtacaaccccatcatctacatcttcatgaatcgacagttcagggactgcgccgtgcctttcctgctgtgcggcaggaatccctgggcagagcccagctccgaatctgccacagcagcctcaaccagcgccacaagtgtgactctggcttcagcaccaggacaggtctccccatcc

SEQ ID NO: 31-32 - skipped (2 entries)

SEQ ID NO: 33 - protein, 342 aa, synthetic construct
  REGION 1..342: note = medakaTMT1A aa sequence
MLVSNVSLGGCAEFNSALCAGAGEEHLGGGSYRTTLTPTGHLIVAVCLGFIGTFGLVNNLLVLVLFCRYKILRSPINLLLINISISDLLVCVLGTPFSFAASTQGRWLIGEGGCVWYGFANSLCGIVSLISLAVLSYERYSTMMTPAEADSSNYRKISLGIILSWGYSLLWTLPPLFGWSHYGPEGPGTTCSVDWTAKTANNISYIICLFVFCLIVPFMVIVFCYGKLLYAIKQVSGINVSVSRKREQRVLFMVVIMVICYLLCWLPYGIMALLATFGPPDLVTPEASIIPSVLAKTSTAINPVIYVFMNKQFFRCFQAMLRCKAPLRGSSARSSSKVATKA

SEQ ID NO: 34 - DNA, 1026 bp, synthetic construct
  misc_feature 1..1026: note = medakaTMT1A na sequence
  source 1..1026: mol_type = other DNA
atgctagtcagtaatgtaagcctgggtgggtgcgccgaattcaattccgctctttgtgcgggcgctggagaagaacacctcggcggtggttcatatcggaccacgctaaccccgactggccatctcattgtcgctgtgtgccttgggttcattggcactttcgggcttgtaaataatttgcttgtcttggtcctattctgtcgttataagattctgcggagccctatcaatcttctacttattaatatcagtatttccgacctgctcgtttgcgtgctgggcacccctttctcctttgctgctagtacacaaggcaggtggttgattggcgagggcggatgtgtatggtacggatttgcgaacagcctgtgtgggatcgttagcctaatttcccttgctgtcctttcttatgaacgttactctactatgatgacccccgcggaagccgactcaagtaattaccggaaaataagtctcggtatcatcctctcctggggctatagtttgctctggacgttgccccctttgtttggctggagtcattacgggcccgaaggaccaggaaccacctgtagcgtcgattggaccgccaagaccgccaataacattagctatattatctgcctgtttgtgttctgtcttatcgtgccgtttatggtgattgtattttgctatggtaaactgctgtacgctattaaacaagtgagcggaattaatgtcagtgtaagtaggaaacgagaacaacgcgtgctctttatggtggtcattatggtcatatgctacctgctttgttggctcccttacggcattatggcccttctcgcaacgtttggacccccagacctcgtcaccccagaagcctctatcataccctcagttctcgcgaagactagtaccgctataaatccagttatctatgtctttatgaataagcagttcttcaggtgtttccaagcaatgcttaggtgtaaagctccactgcgcgggagctcagcaaggtccagctccaaagttgctacaaaagct

SEQ ID NO: 35-36 - skipped (2 entries)

SEQ ID NO: 37 - protein, 341 aa, synthetic construct
  REGION 1..341: note = zPP1 aa sequence
MHEEMESETSTAASGSIAEVMPRTGYTILAVIIGVFSVCGVILNVTVITVTLKYKQLRQPLNFALVNLAVADLGCAVFGGLPTVVTNAMGYFSLGRVGCVLEGFAVAFFGIAALCSVAVIALERCMVVCRPVGSISFQTRHAVFGVAVSWVWSFIWNTPPLFGWGRFELEGVRTSCAPDWYSRDLANVSFIVCYFLLCFALPFSVIVYSYTRLLWTLRQVSRLQVCEGGSAARAEAQVSCMVVVMILAFLLTWLPYASFALCVILIPELYIDPVIATVPMYLTKSSTVFNPIIYIFMNRQFRDRALPFLLCGRNPWAAEAEEEEEETTVSSVSRSTSVSPA

SEQ ID NO: 38 - DNA, 1023 bp, synthetic construct
  misc_feature 1..1023: note = zPP1 na sequence
  source 1..1023: mol_type = other DNA
atgcacgaggaaatggaatcagaaacatctacagcggcctctgggagcattgctgaggtgatgccccgcactggatataccatacttgcagtgattatcggggtcttttcagtctgcggtgtaatactgaacgtaaccgttattaccgtcacacttaaatacaaacagttgcgtcaacctctgaattttgcgctcgttaatctggccgtagcagacctgggatgtgcggtctttggtggccttccaacggttgtcacaaatgccatgggatatttctctttgggccgcgtcggctgcgtactcgaaggattcgctgttgctttcttcggtatcgctgcattgtgcagcgtcgccgtaattgctctcgaacggtgcatggtggtctgcagacccgtggggtcaatcagtttccaaaccagacatgccgtgtttggagttgccgtgagctgggtatggtcatttatctggaacacaccccctctcttcggttggggcaggtttgaacttgagggagtgcggacgagttgcgctccagattggtacagtagagatcttgccaatgtcagctttatagtttgttacttcttgctgtgttttgccttgcctttctctgtgattgtatacagctacactcgtttgctctggacactccgacaagtatctcggctgcaagtatgtgaaggcggttctgcggcacgcgctgaagctcaagtatcctgcatggtagtggtcatgatactcgcttttctcctcacttggctgccatatgcaagtttcgctttgtgtgttatcctcatacctgagctctatatcgaccctgtcatcgccacggtcccaatgtatctcacaaaatcctcaactgtcttcaaccctatcatctatatatttatgaatcggcagtttagggaccgtgctctcccgtttctgctgtgcgggagaaatccatgggctgcggaagccgaggaagaagaagaggaaacaaccgtgtcaagcgtcagcaggtccactagcgtaagccccgcg

SEQ ID NO: 39-40 - skipped (2 entries)

SEQ ID NO: 41 - protein, 336 aa, synthetic construct
  REGION 1..336: note = pPP2 aa sequence
MKPSAFYLNASLYLGPQGEPPLPRSGFIALSVIMALLTGPAIVLNATVIIVSLMHKQLRQPLNYALVNMAVADLGTAMTGGLLSVVNNAQGYFSLGRTGCVLEGFAVSLCGIASLCTVALIAVERMFVICKPLGQMQFQKQHALGGIALAWLWSLTWNLPPLFGWGRYELEGVGTSCAPDWHSREPQNVSYVLAYFTVCFAAPFVIILVSYSKLMWTLHKVTKMACMEGGAVAKSEMTVAYMVILMVVTFLISWLPYAGLSMLVVLSPDVKIHPLVGTVPVYLAKSSTVYNPIIYIYLNKQFRKYAVPFLLCGRELEMEDELSMTTVETSNRVSPA

SEQ ID NO: 42 - DNA, 1008 bp, synthetic construct
  misc_feature 1..1008: note = pPP2 na sequence
  source 1..1008: mol_type = other DNA
atgaagccttctgcattctacctaaatgcaagtctgtacctcgggccacagggagagcccccactgcctcggagtggctttattgctctgtctgtgatcatggctttgctcacaggtccagccatagtcttgaacgctacagtgataatcgtgtccctcatgcataaacaactaaggcagcctttgaattatgcgctcgttaatatggcagtggccgatcttggtacagctatgacgggcggcctgctgtctgtggtgaataatgcgcaaggatactttagcctgggcagaacaggttgcgtactggaaggttttgctgttagtctctgcggcattgcatctctgtgtacggtggcactgatcgccgtcgaacgcatgttcgtcatttgtaaacctctgggccaaatgcaattccaaaagcagcacgcattgggcggcatcgccctggcttggctttggtctctgacatggaatctcccacccctctttgggtggggcagatatgaacttgagggtgtgggcacctcatgcgcgcccgactggcatagccgggagccacaaaatgtatcatacgtccttgcttatttcactgtgtgttttgccgcaccatttgttatcattctggtctcatatagcaaactgatgtggactctgcacaaagtcactaaaatggcttgtatggaaggtggagcagtcgcaaagtctgaaatgaccgtggcctacatggttatcctcatggtagttacattcttgatcagctggctcccctacgccgggctcagcatgctcgtggttctcagcccggatgtgaagatacacccgcttgtgggtactgttcctgtgtacctggctaaatcctctacagtatacaatcccatcatttacatttatttgaacaaacaattccgcaagtacgccgtcccatttctgctctgcggccgggaactcgaaatggaagacgagctttccatgactacagtagaaactagcaatagagttagccctgcc

SEQ ID NO: 43-44 - skipped (2 entries)